(19) United States
(12) Patent Application Publication
(10) Pub. No.: US 2021/0059978 A1
(43) Pub. Date: Mar. 4, 2021

(54) TERPENE-ENRICHED CANNABINOID COMPOSITIONS AND USES THEREOF IN THE TREATMENT OF INFECTIOUS CONDITIONS

(71) Applicant: BUZZELET DEVELOPMENT AND TECHNOLOGIES LTD, Or-Akiva (IL)

(72) Inventors: Noa Raz, Gizo (IL); Aharon M. Eyal, Jerusalem (IL)

(21) Appl. No.: 17/095,799

(22) Filed: Nov. 12, 2020

Related U.S. Application Data
(63) Continuation-in-part of application No. PCT/IB2019/053954, filed on May 13, 2019.
(60) Provisional application No. 62/670,870, filed on May 14, 2018.
(60) Provisional application No. 62/833,671, filed on Apr. 13, 2019.

(51) Int. Cl. A61K 31/352; A61K 31/05; A61K 31/085; A61K 31/015; A61K 31/045; A61K 31/01; A61K 31/125; A61K 31/11; A61K 9/08; A61K 9/00; A61K 9/20; A61K 9/02

(52) U.S. Cl. CPC A61K 31/352; A61K 31/05; A61K 31/085; A61K 31/015; A61K 31/045; A61K 31/01; A61K 9/02; A61K 31/11; A61K 9/08; A61K 9/0014; A61K 9/006; A61K 9/20; A61K 31/125

(57) ABSTRACT

Provided are methods for treating conditions and/or symptoms selected from the group consisting of ulcer, viral infection, bacterial infection, fungal infection, infectious disease, and inflammation comprising administering to a subject in need thereof a therapeutically effective amount of a composition comprising at least one cannabinoid in a specific amount; at least one terpene in a specific amount, and at least 5% by weight of a non-cannabinoid, non-terpene carrier, wherein said non-cannabinoid, non-terpene carrier comprises cellulose and a weight/weight ratio of a total amount of terpenes to a total amount of cannabinoids in said composition is from about 0.1:1 to about 1:1, or wherein said non-cannabinoid, non-terpene carrier comprises less than 5% by weight cellulose and a weight/weight ratio of a total amount of terpenes to a total amount cannabinoids in said composition is about 0.05:1 to about 1:1.

### CROSS-REFERENCE TO RELATED APPLICATIONS

This patent application is a Continuation-in-Part (CIP) of PCT international application number PCT/IB2019/053954, having an international filing date of May 13, 2019, published as international publication number WO 2019/220324 A3, which is hereby incorporated by reference in its entirety; which claims benefit and priority from U.S. 62/670,870, filed on May 14, 2018 and U.S. 62/833,671, filed on Apr. 13, 2019, which are hereby incorporated by reference in their entirety.

### FIELD OF THE INVENTION

The field of art to which this invention generally pertains is cannabinoid compositions, and specifically to cannabinoid compositions for use in the treatment of ulcer, viral infection, bacterial infection, fungal infection, infectious disease, and inflammation.

### BACKGROUND OF THE INVENTION

An infection is the invasion of an organism's body tissues by disease-causing agents, their multiplication, and the reaction of host tissues to the infectious agents and the toxins they produce. Infectious disease is illness resulting from an infection. Infections are caused by infectious agents including viruses, viroids prions, bacteria, nematodes such as parasitic roundworms and pinworms, arthropods such as ticks, mites, fleas, and lice, fungi such as ringworm, and other macroparasites such as tapeworms and other helminths.

An ulcer is a discontinuity or break in a bodily membrane that impedes the organ of which that membrane is a part from continuing its normal functions. Several types of ulcers, including pressure and/or diabetes ulcers are very hard to cure.

Conditions and/or symptoms associated with an infection, infectious diseases and/or ulcer may include local pain, itch and burn.

All the above can be treated by a localized topical treatment, instead of or in addition to a systemic treatment.

Localized topical treatment may be preferable also for treating pain, itch and/or burn from other sources.

Topical treatment refers to administrating a composition on top of the site of infection, pain, burn and/or itch. Topical treatments are efficient in mainly conditions, including dermal and oral cavity.

Terpenes play important roles in cannabinoids-comprising products, affecting the functionality and bioavailability of the cannabinoids and the aroma of the product. Processing cannabis plant material typically leads to terpenes loss so that most of cannabis products are of relatively low terpene content. That is particularly true for cannabis extracts and products thereof, such as cannabis tablets, cannabis gel capsules, cannabis patches, cannabis suppositories, etc. Most cannabis terpenes boiling points are in the range between about 150° C. and about 220° C. and they evaporate, at least partially, during cannabis buds drying, during solvent separation from extracts and during decarboxylation. Monoterpenes are lost at a rate greater than that of terpenes with a higher molecular weight and terpenes carrying no hydroxyl groups are lost at a rate greater than that of terpenes that do carry hydroxyl groups.

### SUMMARY OF THE INVENTION

According to an aspect of some embodiments of the present invention, there is provided a method for treating conditions and/or symptoms selected from the group consisting of ulcer, viral infection, bacterial infection, fungal infection, infectious disease, and inflammation comprising administering to a subject in need thereof a therapeutically effective amount of a composition comprising
i. at least one cannabinoid in a specific amount,
ii. at least one terpene in a specific amount, and
iii. at least 5% by weight of a non-cannabinoid, non-terpene carrier
   a. wherein said non-cannabinoid, non-terpene carrier comprises cellulose and a weight/weight ratio of a total amount of terpenes to a total amount of cannabinoids in said composition is from about 0.1:1 to about 1:1, or;
   b. wherein said non-cannabinoid, non-terpene carrier comprises less than 5% by weight cellulose and a weight/weight ratio of a total amount of terpenes to a total amount cannabinoids in said composition is about 0.05:1 to about 1:1.

According to some embodiments, said conditions and/or symptoms are selected from the group consisting of fungal infection, dermatophytes, candida infection, athlete's foot (Tinea pedis), yeast infection, jock itch (Tinea cruris), ringworm (Tinea Corporis), Tinea Capitis, Onychomycosis, Tinea Versicolor, Tinea Cruris, viral infection, Herpes, Herpes simplex, Herpes Zoster, Hand Foot and Mouth disease, Molluscum contagiosum, Viral Warts, Chickenpox, Measles, Rubella, bacterial infection, acne, Methycillin-Resistant Staphylococcus Aureus (MRSA), Necrotizing Fasciitis, Staphylococcal Scalded Skin Syndrome (SSSS), Leprosy, Cellulitis, Erysipelas, ulcer, pressure ulcer, diabetic ulcer, oral ulcer, mucositis, dermatitis, eczema, seborrhea, Lichen planus, periodontal disease, leishmaniasis, scabies, malaria, an inflammatory injury, psoriasis, local pain, local itch and/or local burn, Temporomandibular-Joint (TMJ) and combination thereof.

According to some embodiments, said at least one cannabinoid is selected from the group consisting of CBD, THC, CBC, CBG, CBN, CBL, CBDV, THCV and combinations thereof, wherein said at least one terpene is selected from the group consisting of pinene, limonene, caryophyllene, caryophyllene oxide, nerolidol, eucalyptol, terpinene, terpineol, myrcene, cycloartenol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral and combinations thereof and wherein said conditions and/or symptoms comprise a fungal infection.

According to some embodiments, said at least one cannabinoid is selected from the group consisting of CBD, THC, CBC, CBG, CBN, CBL, CBDV, THCV and combinations thereof, wherein said at least one terpene is selected from the group consisting of pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral and combinations thereof and wherein said conditions and/or symptoms comprise Candida infection.

According to some embodiments, said at least one cannabinoid is selected from the group consisting of CBD, THC, CBC, CBG, CBN, CBL, CBDV, THCV and combinations thereof, wherein said at least one terpene is selected from the group consisting of sabine, eucalyptol, pinene, borneol, caryophyllene, geraniol, limonene, myrcene, cycloartenol, citronellol, humulene, linalool, eudesmol, camphor, menthol and combinations thereof and wherein said conditions and/or symptoms comprise a viral infection.

According to some embodiments, said at least one cannabinoid is selected from the group consisting of CBD, THC, CBC, CBG, CBN, CBDa and THCa, CBLA, THCV and combinations thereof, wherein said at least one terpene is selected from the group consisting of geraniol, eucalyptol, pinene, borneol, caryophyllene, terpineol, limonene, myrcene, cycloartenol, citronellol, humulene, linalool, eudesmol, camphor, menthol and combinations thereof and wherein said conditions and/or symptoms comprise herpes, optionally herpes simplex, and/or herpes zoster.

According to some embodiments, said at least one cannabinoid is selected from the group consisting of CBD, THC, CBC, CBG, CBN, CBL, CBDV, THCV and combinations thereof, wherein said at least one terpene is selected from the group consisting of eucalyptol, citronellol, pinene, borneol, caryophyllene, geraniol, limonene, myrcene, cycloartenol, citronellol, humulene, linalool, eudesmol, camphor, menthol and combinations thereof and wherein said conditions and/or symptoms comprise Hand Foot and Mouth Disease.

According to some embodiments, said at least cannabinoid is selected from the group consisting of CBD, THC, CBC, CBG, CBN, CBL, CBDV, THCV and combinations thereof, wherein said at least one terpene is selected from the group consisting of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, fenchol, geranyl acetate, camphor, menthol and combinations thereof and wherein said conditions and/or symptoms comprise a bacterial infection.

According to some embodiments, said at least one cannabinoid is selected from the group consisting of CBD, THC, CBC, CBG, CBN, CBL, CBDV, THCV and combinations thereof, wherein said at least one terpene is selected from the group consisting of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, camphor, menthol and combinations thereof and wherein said conditions and/or symptoms comprise acne.

According to some embodiments, said at least one cannabinoid is selected from the group consisting of CBD, THC, CBC, CBG, CBN, CBL, CBDV, THCV and combinations thereof, wherein said at least one terpene is selected from the group consisting of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, camphor, menthol and combinations thereof and wherein said conditions and/or symptoms comprise Methycillin-Resistant Staphylococcus Aureus (MRSA).

According to some embodiments, said at least one cannabinoid is selected from the group consisting of CBD, THC, CBC, CBG, CBN, CBL, CBDV, THCV and combinations thereof, wherein said at least one terpene is selected from the group consisting of pinene, limonene, linalool, germacrene, caryophyllene, nerolidol, terpineol, terpinene, eucalyptol, myrcene, cirtonellol, humulene, eudesmol, camphor, menthol and combinations thereof and wherein said conditions and/or symptoms comprise an ulcer, optionally a pressure ulcer and/or diabetic ulcer.

According to some embodiments, said at least one cannabinoid is selected from the group consisting of CBD, THC, CBC, CBG, CBN, CBL, CBDV, THCV and combinations thereof, wherein said at least one terpene is selected from the group consisting of pinene, limonene, linalool, germacrene, caryophyllene, nerolidol, terpineol, terpinene, eucalyptol, myrcene, cirtonellol, humulene, eudesmol, camphor, menthol and combinations thereof and wherein said conditions and/or symptoms comprise oral ulcer and/or mucositis.

According to some embodiments, said at least one cannabinoid is selected from the group consisting of CBD, THC, CBC, CBG, CBN, CBL, CBDV, THCV and combinations thereof, wherein said at least one terpene is selected from the group consisting of camphene, sabinene, eugenol, cirtonellol, gerniol, terpineol, pinene, limonene, linalool, germacrene, caryophyllene, nerolidol, terpinene, eucalyptol, myrcene, humulene, eudesmol, camphor, menthol and combinations thereof and wherein said conditions and/or symptoms are selected from the group consisting of dermatitis, eczema, and seborrhea and combinations thereof.

According to some embodiments, said at least one cannabinoid is selected from the group consisting of CBD, THC, CBC, CBG, CBN, CBL, CBDV, THCV and combinations thereof, wherein said at least one terpene is selected from the group consisting of pinene, limonene, nerolidol, Cycloartenol, caryophyllene, caryophyllene oxide, amyrin, eucalyptol, myrcene, cirtonellol, humulene, linalool, farnesene, phellandrene, Cymenen, eudesmol, carvacrol, eugenol, camphor, menthol and combinations thereof and wherein said conditions and/or symptoms comprise a periodontal disease.

According to some embodiments, said at least one cannabinoid is selected from the group consisting of CBD, THC, CBC, CBG, CBN, CBL, CBDV, THCV and combinations thereof, wherein said at least one terpene is selected from the group consisting of linalool, nerolidol, pinene, limonene, germacrene, caryophyllene, terpineol, terpinene, eucalyptol, myrcene, cirtonellol, humulene, eudesmol, camphor, menthol and combinations thereof and wherein said conditions and/or symptoms comprise leishmaniasis.

According to some embodiments, said at least one cannabinoid is selected from the group consisting of CBD, THC, CBC, CBG, CBN, CBL, CBDV, THCV and combinations thereof, wherein said at least one terpene is selected from the group consisting of caryophyllene, nerolidol, pinene, eucalyptol, limonene, myrcene, cirtonellol, humulene, terpineol, terpinene, guaiol, friedelin, eudesmol, linalool, camphor, menthol and combinations thereof and wherein said conditions and/or symptoms comprise malaria.

According to some embodiments, said at least one cannabinoid is selected from the group consisting of CBD, THC, CBC, CBG, CBN, CBL, CBDV, THCV and combinations thereof, wherein said at least one terpene is selected from the group consisting of caryophyllene, nerolidol, pinene, eucalyptol, limonene, myrcene, cirtonellol, humulene, terpineol, terpinene, guaiol, friedelin, eudesmol, linalool, camphor, menthol and combinations thereof and wherein said conditions and/or symptoms comprise scabies.

According to some embodiments, said at least one cannabinoid is selected from the group consisting of CBD, THC, CBC, CBG, CBN, CBL, CBDV, THCV and combinations thereof, wherein said at least one terpene is selected from the group consisting of pinene, limonene, eucalyptol, caryophyllene myrcene, citronellol, humulene, terpinene, terpineol, guaiol, friedelin, eudesmol linalool, borneol, amyrin, cycolartenol, camphor, menthol and combinations thereof and wherein said conditions and/or symptoms are selected from the group consisting of an inflammatory injury, wound, sore and ulcer.

According to some embodiments, said at least one cannabinoid is selected from the group consisting of CBD, THC, CBC, CBG, CBN, CBL, CBDV, THCV and combinations thereof, wherein said at least one terpene is selected from the group consisting of linalool, caryophyllene, limonene, eucalyptol, myrcene, cirtonellol, pinene, sabinene, terpineol, terpinene, camphor, menthol and combinations thereof and wherein said conditions and/or symptoms comprise psoriasis.

According to some embodiments, said at least one cannabinoid is selected from the group consisting of CBD, THC, CBC, CBG, CBN, CBL, CBDV, THCV and combinations thereof, wherein said at least one terpene is selected from the group consisting of myrcene, linalool, caryophyllene, eucalyptol, borneol, pinene, sabinene, terpineol, camphor, menthol and combinations thereof and wherein said conditions and/or symptoms are selected from the group consisting of local pain, local itch and local burn and combinations thereof.

According to some embodiments, the method comprises administering to said subject for a first period of time a first said composition comprising a first cannabinoid at a first cannabinoid amount and a first primary terpene at a first primary terpene amount, followed by administering to said subject for a second period of time a second said composition comprising said first cannabinoid at said first cannabinoid amount and a second primary terpene at a second primary terpene amount.

According to some embodiments, said condition and/or symptoms are selected from the group consisting of a fungal infection, bacterial infection, inflammation and ulceration, said composition comprising at least one cannabinoid at a concentration of about 10-50 mg/L, wherein said preparation comprises an aqueous medium.

### DETAILED DESCRIPTION OF THE INVENTION

Unless indicated otherwise, the term “infection” as used herein refers to the invasion of an organism's body tissues by disease-causing agents, their multiplication, and the reaction of host tissues to the infectious agents and the toxins they produce.

Unless indicated otherwise, the term “infectious disease” as used herein refers to illness resulting from an infection.

As used herein the term “treating” includes preventing, curing, ameliorating, mitigating, and reducing the instances or severity of a condition or a symptom thereof.

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

Unless indicated otherwise, percent is weight percent and ratio is weight/weight ratio. Unless indicated otherwise, weight ratio means the ratio between weight content, e.g. in an aqueous solution containing 20% solute and 80% water, the solute to water weight ratio is 20:80 or 1:4.

Unless indicated otherwise, the term “cannabinoid” as used herein refers to a compound that affects the endocannabinoid system. Cannabinoids are agonists or antagonists to receptors in the endocannabinoid system.

As used herein, the term “THC” refers to THCa (tetrahydrocannabiniolic acid) and/or to THC (tetrahydrocannabiniol) unless indicated otherwise. As used herein, the term “CBD” refers to CBDa (cannabidiolic acid) and/or to CBD (cannabidiol) unless indicated otherwise. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise. As used herein, the term “CBL” refers to CBLa (Cannabicycol acid) and/or to CBL (Cannabicyclol) unless indicated otherwise. As used herein, the term “THCV” refers to THCVa (tetrahydrocannabivarin acid) and/or to THCV (tetrahydrocannabivarin) unless indicated otherwise. As used herein, the term “CBDV” refers to CBDVa (cannabigerovarin acid) and/or to CBDV (cannabidivarin) unless indicated otherwise.

As used herein, the term “cellulose” refers to cellulose, hemicellulose and their combinations. As used herein, the term “glycol” refers to any glycol, including ethylene glycol, polyethylene glycol, propylene glycol and polypropylene glycol. As used herein, the term “chlorophyll” refers to chlorophyll and degradation products thereof.

As used herein, “terpene” refers to both terpenes and terpenoids.

As used herein and unless indicated otherwise, the term “terpenes/cannabinoids (or terpenes to cannabinoids) weight/weight ratio” means the weight ratio between the combined amounts of terpenes and the combined amounts of cannabinoids.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an embodiment, provided is a terpene-enriched cannabinoid composition comprising (i) at least one cannabinoid in a specific amount, (ii) at least one primary terpene in a specific amount, (iii) at least 5% by weight of a non-cannabinoid, non-terpene, carrier, (iv) optionally at least two secondary terpenes, and (a) wherein the non-cannabinoid, non-terpene carrier comprises cellulose and a weight/weight ratio of a total amount of terpenes to a total amount of cannabinoids in the composition is from about 0.1:1 to about 1:1, or (b) wherein the non-cannabinoid, non-terpene carrier comprises less than 5% by weight cellulose and a weight/weight ratio of a total amount of terpenes to a total amount of cannabinoids in the composition is from about 0.05:1 to about 1:1.

According to an embodiment, provided is a terpene-enriched cannabinoid composition for treating conditions and/or symptoms associated with at least one selected from the group consisting of ulcer, viral infection, bacterial infection, fungal infection, infectious disease and inflammation and combinations thereof, the composition comprising (i) at least one cannabinoid in a specific amount, (ii) at least one primary terpene in a specific amount, (iii) at least 5% by weight of a non-cannabinoid, non-terpene, carrier, (iv) optionally at least two secondary terpenes, and (a) wherein the non-cannabinoid, non-terpene carrier comprises cellulose and a weight/weight ratio of a total amount of terpenes to a total amount of cannabinoids in the composition is from about 0.1:1 to about 1:1, or (b) wherein the non-cannabinoid, non-terpene carrier comprises less than 5% by weight cellulose and a weight/weight ratio of a total amount of terpenes to a total amount of cannabinoids in the composition is from about 0.05:1 to about 1:1.

According to an embodiment, the composition has a therapeutic effect in treating conditions and/or symptoms associated with at least one selected from the group consisting of ulcer, viral infection, bacterial infection, fungal infection, infectious disease and inflammation and combinations thereof, wherein the therapeutic effect is an enhanced therapeutic effect compared with that of a composition comprising the same cannabinoids amounts and one fifth the amount of at least one primary terpene. According to an embodiment, the composition has a therapeutic effect in treating at least one conditions and/or symptom selected from the group consisting of ulcer, viral infection, bacterial infection, fungal infection, infectious disease and inflammation and combinations thereof, wherein the therapeutic effect is an enhanced therapeutic effect compared with that of a composition comprising the same cannabinoids amounts and one half, one third, one quarter or one fifth the amount of at least one primary terpene.

According to an embodiment, the conditions and/or symptoms are selected from the group consisting of skin ulcer, skin viral infection, skin bacterial infection, skin fungal infection, skin infectious disease and skin inflammation and combinations thereof.

According to an embodiment, the composition is for topical application.

According to an embodiment, the conditions and/or symptoms are selected from the group consisting of fungal infection, dermatophytes, candida infection, athlete's foot (Tinea pedis), yeast infection, jock itch (Tinea cruris), ringworm (Tinea Corporis), Tinea Capitis, Onychomycosis, Tinea Versicolor, Tinea Cruris, viral infection, Herpes, Herpes simplex, Herpes Zoster, Hand Foot and Mouth disease, Molluscum contagiosum, Viral Warts, Chickenpox, Measles, Rubella, bacterial infection, acne, Methycillin-Resistant Staphylococcus Aureus (MRSA), Necrotizing Fasciitis, Staphylococcal Scalded Skin Syndrome (SSSS), Leprosy, Cellulitis, Erysipelas, ulcer, pressure ulcer, diabetic ulcer, oral ulcer, mucositis, dermatitis, eczema, seborrhea, Lichen planus, periodontal disease, leishmaniasis, scabies, an inflammatory injury, psoriasis, local pain, local itch and/or local burn, Temporomandibular-Joint (TMJ) and combination thereof.

According to an embodiment, the conditions and/or symptoms are selected from the group consisting of fungal infection, athlete's foot (Tinea pedis), yeast infection, jock itch (Tinea cruris), ringworm (Tinea Corporis), Tinea Capitis, Onychomycosis, Tinea Versicolor, Tinea Cruris, viral infection, Herpes, Herpes simplex, Herpes Zoster, Hand Foot and Mouth disease, Molluscum contagiosum, Viral Warts, Chickenpox, Measles, Rubella, bacterial infection, Necrotizing Fasciitis, Staphylococcal Scalded Skin Syndrome (SSSS), Leprosy, Cellulitis, Erysipelas, ulcer, pressure ulcer, diabetic ulcer, oral ulcer, mucositis, dermatitis, eczema, seborrhea, Lichen planus, periodontal disease, Scabies, an inflammatory injury, local pain, Temporomandibular-Joint (TMJ) and combinations thereof.

According to an embodiment, the conditions and/or symptoms are selected from the group consisting of fungal infection, athlete's foot (Tinea pedis), yeast infection, jock itch (Tinea cruris), ringworm (Tinea Corporis), Tinea Capitis, Onychomycosis, Tinea Versicolor, Tinea Cruris, viral infection, Herpes, Herpes simplex, Herpes Zoster, Hand Foot and Mouth disease, Molluscum contagiosum, Viral Warts, Chickenpox, Measles, Rubella, bacterial infection, Necrotizing Fasciitis, Staphylococcal Scalded Skin Syndrome (SSSS), Leprosy, Cellulitis, Erysipelas, ulcer, pressure ulcer, diabetic ulcer, oral ulcer, mucositis, dermatitis, eczema, seborrhea, Lichen planus, periodontal disease, Scabies, an Temporomandibular-Joint (TMJ) and combinations thereof.

According to an embodiment, the increased therapeutic effect comprises an increased immune response.

According to an embodiment, the composition comprises at least one cannabinoid, at least two cannabinoids, at least three, at least four or at least five. According to an embodiment, the content of each cannabinoid in the composition is at least 10 parts per million (ppm). As known in the art, cannabinoids have an acid form and a non-acid form (which is also referred to as decarboxylated form, since it can be generated by decarboxylating the acid form). The acid form is indicated herein by the letter (a) at the end of the cannabinoid acronym, e.g. tetrahydrocannabiniolic acid is indicated as THCa, while the decarboxylated form is THC. According to an embodiment, the cannabinoids are selected from the group consisting of tetrahydrocannabiniol in acid or decarboxylated form (THCa or THC, respectively), cannabidiol in acid or decarboxylated form (CBDa or CBD, respectively), cannabigerol in acid or decarboxylated form (CBGa or CBG, respectively), cannabichromene in acid or decarboxylated form (CBCa or CBC, respectively) tetrahydrocannabivarin in acid or decarboxylated form (THCVa or THCV, respectively), Cannabidivarin in acid or decarboxylated form (CBDVa or CBDV respectively) and cannabinol in acid or decarboxylated form (CBNa or CBN, respectively), Cannabicyclol in acid or decarboxylated form (CBLa or CBL, respectively). As used herein, the term THC refers to THCa (tetrahydrocannabiniolic acid) and/or to THC (tetrahydrocannabiniol) unless indicated otherwise. As used herein, the term CBD refers to CBDa (tcannabidiolic acid) and/or to CBD (cannabidiol) unless indicated otherwise. Thus, the term “CBD to THC ratio” may mean “CBD to THC ratio”, “CBDa to THC ratio”, “CBD to THCa ratio”, “CBDa to THCa ratio”, “CBD to THC+THCa ratio”, “CBDa to THC+THCa ratio”, “CBD+CBDa to THC ratio”, “CBD+CBDa to THCa ratio” or “CBD+CBDa to THC+THCa ratio”. As used herein, the term CBG refers to CBGa (cannabigerolic acid) and/or to CBG (cannabigerol) unless indicated otherwise. As used herein, the term CBN refers to CBNa (Cannabinolic acid) and/or to CBN (cannabinol) unless indicated otherwise. As used herein, the term CBC refers to CBCa (cannabichromenic acid) and/or to CBC (Cannabichromene) unless indicated otherwise. As used herein, the term CBL refers to CBLa (Cannabicycol acid) and/or to CBL (Cannabicyclol) unless indicated otherwise.

According to an embodiment, at least one of the cannabinoids is in acid form. According to an embodiment, at least one of the cannabinoid is at least partially in decarboxylated form. According to an embodiment, at least 50% of the cannabinoid is in decarboxylated form, at least 60%, at least 70%, at least 80% or at least 90%.

According to an embodiment, the composition comprises THC and/or THCa. According to an embodiment, the composition comprises CBD and/or CBDa. According to an embodiment, the composition comprises THC and/or THCa at a content of less than 1%, less than 0.8%, less than 0.6%, less than 0.4% or less than 0.2%. According to an embodiment, the composition comprises both CBD and/or CBDa and THC and/or THCa and the weight/weight ratio between CBD and/or CBDa and THC and/or THCa ((CBD+CBDa)/(THC+THCa)) is at least 10, at least 15, at least 20, at least 25 or at least 30. According to an embodiment, the composition comprises CBG and/or CBGa. According to an embodiment, the composition comprises CBN and/or CBNa. According to an embodiment, the composition comprises CBC and/or CBCa. According to an embodiment, the composition comprises CBL and/or CBLa. According to an embodiment, the composition comprises THCV and/or THCVa. According to an embodiment, the composition comprises CBDV and/or CBDVa.

According to an embodiment, the composition comprises at least 2% by weight carrier, at least 3%, at least 5%, at least 10%, at least 15%, at least 20%, at least 25%, at least 30%, at least 35% or at least 40%, at least 50%, at least 60%, at least 70%, or at least 80% by weight carrier. Any compound other than cannabinoids and terpenes is a suitable carrier. According to an embodiment, the carrier is selected from the group consisting of vegetable oils, e.g. coconut oil, olive oil or sesame oil, pharmaceutical excipients, honey, bees wax, cellulose and combinations thereof. As used herein, the term cellulose refers to cellulose, hemicellulose and their combinations.

According to an embodiment, the composition comprises less than 5% by weight glycol, less than 4%, less than 3%, less than 2%, or less than 1%, by weight glycol. As used herein, the term glycol refers to any glycol, including ethylene glycol, polyethylene glycol, propylene glycol and polypropylene glycol.

According to an embodiment, the composition comprises water and water content is less than 30% by weight, less than 25%, less than 20%, less than 15%, less than 12%, less than 10%, or less than 8%. According to an embodiment, water content is at least 1% by weight, at least 2%, at least 3%, at least 4% or at least 5%.

According to an embodiment, the composition comprises at least one primary terpene and optionally at least two secondary terpenes, at least three, at least four or at least five. The term “terpene”, as used herein, refers to both terpenes and terpenoids. As used here, the term “primary terpene” refers to a terpene that forms at least 20% by weight of the total amount of terpenes in the composition, at least 30%, at least 40%, at least 50%, at least 60%, at least 70% or at least 8%. According to an embodiment, the composition comprises multiple (e.g. two or three) terpenes, each one of which forms at least 20% by weight of the total amount of terpenes in the composition and each one of these terpenes is considered a primary terpene. As used here, the term “secondary terpene” refers to a terpene that forms at least 10 parts per million (ppm) of the composition. According to an embodiment, the content of the at least one primary terpene in the composition is at least 2 times greater than that of any secondary terpene, at least 4, at least 6, at least 8, at least 10, at least 15, at least 20, at least 25, or at least 30 times greater.

As used herein, “terpenes/cannabinoids (or terpenes to cannabinoids) weight/weight ratio” means the weight ratio between the combined amount of terpenes and the combined amount of cannabinoids. According to an embodiment, the composition comprises at least 5% by weight cellulose and the terpenes to cannabinoids weight/weight ratio in the composition is in the range between about 0.1 and about 1.0. According to an embodiment, the ratio is greater than 0.1, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 0.9, less than 0.8, less than 0.7, less than 0.6, less than 0.5, less than 0.4, less than 0.3, less than 0.2 or less than 0.15. According to an embodiment, the composition comprising more than 5% cellulose is selected from the group consisting of cannabis plant material, e.g. cannabis buds or cannabis trim, ground forms thereof, plant material preparation for vaporizers and cannabis cigarette. According to an embodiment, the composition comprises a dried cannabis plant material.

According to another embodiment, the non-cannabinoid, non-terpene carrier comprises less than 5% by weight cellulose and terpenes to cannabinoids weight/weight ratio in the composition is in the range between about 0.05 and about 1.0. According to an embodiment, the ratio is greater than 0.1, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 0.9, less than 0.8, less than 0.7, less than 0.6, less than 0.5, less than 0.4, less than 0.3, less than 0.2, less than 0.15 or less than 0.1. According to an embodiment, the composition comprising less than 5% cellulose is selected from the group consisting of cannabis trichomes, cannabis extracts and products thereof, such as tablets, gel capsules, medical patches, vaporizer liquids, mouthwashes, varnishes and suppositories.

According to an embodiment, the at least one primary terpene, the secondary terpenes or both are selected from the group consisting of pinene, limonene, linalool, caryophyllene, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, geranyl acetate, camphor, menthol, iso-menthone, neral, gerial, viridiflorol, germacrene, thymol, Menth-2-en-1-ol, farensol, carotol, myrtenol isomers thereof and combinations thereof. According to an embodiment, the at least one primary terpene the secondary terpenes or both, are selected from the group consisting of pinene, limonene, linalool, caryophyllene, myrcene, terpinene, terpineol and combinations thereof. According to an embodiment, at least one of the terpenes is a-cyclic. According to an embodiment, at least one of the terpenes is cyclic. According to an embodiment, at least one of the terpenes is not found in cannabis buds or is present there at less than 0.2%, less than 0.1%, less than 0.05% or less than 0.02%. Such terpene is referred to as “non-cannabis terpene”. According to an embodiment, the terpene-enriched cannabinoid composition comprises the non-cannabis terpene at a concentration of at least 0.2%, at least 0.5%, least 0.8%, at least 1%, least 1.5%, at least 2%, at least 3%, least 4%, at least 5%, at least 8% or at least 12%.

According to an embodiment, the at least one primary terpene is selected from the group consisting of pinene, limonene, linalool, caryophyllene, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, isopulegol, guaiol, phellandrene, eudesmol, ocimene, cardinene, elemene, gurjunene, farnesene, friedelin, carvacrol, eugenol, fenchol, geranyl acetate, camphor, menthol, iso-menthone, neral, gerial, viridiflorol, germacrene, thymol, Menth-2-en-1-ol, farensol, carotol, myrtenol and combinations thereof. According to an embodiment, the at least one primary terpene is selected from the group consisting of pinene, limonene, linalool, caryophyllene, myrcene and humulene and combinations thereof. According to an embodiment, the at least one primary terpene is a non-cannabis terpene.

According to an embodiment, the terpenes comprise at least one monoterpene selected from the group consisting of limonene, myrcene, pinene, linalool, geraniol, terpinolene camphene and isomers thereof and combinations thereof. According to an embodiment, the terpenes comprise at least one sesquiterpene selected from the group consisting of nerolidol, caryophyllene, farnesene, zingiberene, vetivazulene, guaiazulene, longifolene, copaene, patchoulol humulene and isomers thereof and combinations thereof. According to an embodiment, the terpenes comprise at least one diterpene selected from the group consisting of phytol, retinal, retinol, phytane, cembrene, sclarene, labdane, abietane, texadiene, stemarene, stemoden and isomers thereof and combinations therefo. According to an embodiment, the terpenes comprise at least one hydroxy-terpene selected from the group consisting of nerolidol, geraniol, linalool, phytol and isomers thereof and combinations thereof. As used herein “hydroxy-terpene” refers to a terpene carrying a hydroxyl function.

According to an embodiment, at least one of the terpenes is a monoterpene, at least one of the terpenes is a sesquiterpene and the monoterpenes to sesquiterpenes weight/weight ratio (i.e. the weight ratio between the total amount of monoterpenes and the total amount of the sesquiterpenes) is greater than 1.5 greater than 2, greater than 2.5, greater than 3, greater than 3.5, greater than 4, greater than 4.5, greater than 5, greater than 6, greater than 7, greater than 8, greater than 9, greater than 10, greater than 15, or greater than 20.

According to an embodiment, at least one of the terpenes is a monoterpene, at least one of the terpenes is a diterpene and the monoterpenes to diterpenes weight/weight ratio (i.e. the weight ratio between the total amount of monoterpenes and the total amount of the diterpenes) is greater than 5, greater than 6, greater than 7, greater than 8, greater than 9, greater than 10, greater than 12, greater than 14, greater than 16, greater than 18, greater than 20, greater than 25, or greater than 30.

According to an embodiment, at least one of the terpenes carries no hydroxyl group, at least one of the terpenes carries hydroxyl group and the non-hydroxy-terpenes to hydroxyl-terpenes weight/weight ratio (i.e. the weight ratio between the total amount of non-hydroxy-terpenes and the total amount of the hydroxy-terpenes) is greater than 1.5, greater than 2, greater than 2.5, greater than 3, greater than 3.5, greater than 4, greater than 4.5, greater than 5, greater than 6, greater than 7, greater than 8, greater than 9, greater than 10, greater than 15, or greater than 20.

According to an embodiment, terpenes form at least 20% by weight of the composition, at least 30%, at least 40%, at least 50%, at least 60% or at least 70%.

According to an embodiment, the composition is liquid at 30° C. According to an embodiment, the composition is a suspension at 30° C. According to an embodiment, the composition is essentially clear of haze or suspended solids at 30° C.

According to an embodiment, the composition comprises cannabis plant material. According to an embodiment, the composition comprises cannabis bud. According to an embodiment, the cannabis bud forms at least 20% of the composition, at least 30%, at least 40%, at least 50%, at least 60%, at least 70% or at least 80%.

According to an embodiment the composition comprises tetrahydrocannabinol (THC) and/or tetrahydrocannabinolic acid (THCa), wherein THC and/or THCa is in a total concentration of at least 1% by weight, at least 2%, at least 3%, at least 4%, at least 5%, at least 6%, at least 8%, at least 10%, at least 12%, at least 14%, at least 16%, at least 18%, or at least 20%, at least 30%, at least 40%, at least 50%, at least 60%, at least 70%, at least 80%, or at least 90% by weight.

According to an embodiment, the composition comprises cannabidiol (CBD) and/or cannabidiolic acid (CBDa), wherein CBD and/or CBDa is in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the composition comprises tetrahydrocannabinol (THC) and/or tetrahydrocannabinolic acid (THCa) and cannabidiol (CBD) and/or cannabidiolic acid (CBDa), wherein, THC and/or THCa is in a total concentration of at least 2.5% by weight, and CBD and/or CBDa is in a total concentration of at least 2.5% by weight; THC and/or THCa in a total concentration of at least 3% by weight, and CBD and/or CBDa in a total concentration of at least 3% by weight; THC and/or THCa in a total concentration of at least 4% by weight, and CBD and/or CBDa in a total concentration of at least 4% by weight; THC and/or THCa in a total concentration of at least 5% by weight, and CBD and/or CBDa in a total concentration of at least 5% by weight; THC and/or THCa in a total concentration of at least 8% by weight, and CBD and/or CBDa in a total concentration of at least 8% by weight; THC and/or THCa in a total concentration of at least 10% by weight, and CBDc and/or CBDa in a total concentration of at least 10% by weight.

According to a related embodiment, the composition comprises cannabigerol (CBG) and/or cannabigerol acid (CBGa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises cannabinol (CBN) and/or cannabinol acid (CBNa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises cannabichromene (CBC) and/or cannabichromenic acid (CBCa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises cannabicyclol (CBL) and/or cannabicyclol acid (CBLa) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises tetrahydrocannabivarin (THCV), and/or tetrahydrocannabivarin acid (THCVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises cannabidivarin (CBDV) and/or cannabigerovarin acid (CBGVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the composition comprises less than 5% by weight glycol, less than 4%, less than 3%, less than 2%, or less than 1%, by weight glycol. As used herein, the term glycol refers to any glycol, including ethylene glycol, polyethylene glycol, propylene glycol and polypropylene glycol.

According to an embodiment, the composition comprises water and water content is less than 30% by weight, less than 25%, less than 20%, less than 15%, less than 12%, less than 10%, or less than 8%. According to an embodiment, water content is at least 1% by weight, at least 2%, at least 3%, at least 4% or at least 5% by weight. According to an embodiment, the composition comprises water and water content is more than 50%, more than 60%, more than 70% more than 80%, more than 90%, or more than 95%.

According to an embodiment, the composition comprises chlorophyll. According to an embodiment, the composition comprises at least 0.5% chlorophyll, at least 1, % at least 5%, at least 10%, at least 15%, at least 20% chlorophyll. As used herein, the term chlorophyll refers to chlorophyll and degradation products thereof. According to an embodiment, the composition comprises at least one flavonoid. According to an embodiment, the composition comprises at least two, at least three, at least four, or at least five flavonoids. According to an embodiment, the composition comprises at least one of bergamottin, apigenin, amentoflavone, quercetin and piperine. According to an embodiment, the composition comprises at least two, at least three, or at least four of bergamottin, apigenin, amentoflavone, quercetin and piperine. According to an embodiment, the composition comprises bergamottin. According to an embodiment, the composition comprises apigenin. According to an embodiment, the composition comprises amentoflavone. According to an embodiment, the composition comprises quercetin. According to an embodiment, the composition comprises piperine.

According to an embodiment, the composition comprises a dried cannabis plant material. According to an embodiment, the composition comprises ground cannabis plant material, is a vaporizer cannabis filling material or is a cannabis cigarette.

According to an embodiment, the composition is liquid. According to an embodiment, the composition is selected from the group consisting of vaporizer cannabis filling liquid, cannabis tablets, cannabis suppositories, cannabis gel capsules, cannabis candies, cannabis drinks, cannabis mouthwashes, cannabis varnishes and cannabis baked products.

According to an embodiment, the composition further comprises an additive selected from the group consisting of antioxidants, emulsifiers and texturizers vegetable oils, plant extracts, honey, pharmaceutical excipients, sucrose, glucose and fructose, pharmaceutical excipients and combinations thereof. According to an embodiment, the composition comprises a surfactant selected from the group consisting of phospholipids, glycerides, glycolipids and combinations thereof. According to an embodiment, the composition further comprises a food-approved texturizer. According to an embodiment, the composition further comprises at least ioppm ethanol. According to an embodiment, the composition further comprises at least one of vitamin C, vitamin E, polyunsaturated fatty acids, beeswax and coconut oil. According to an embodiment, the composition further comprises a sweetener.

According to an embodiment, the composition is selected from the group consisting of cannabis tablets, cannabis gel capsules, cannabis medical patches, cannabis cigarettes and cannabis vaporizer liquids, cannabis suppositories, cannabis candies, cannabis drinks, cannabis mouthwashes, cannabis varnishes and cannabis baked products.

According to an embodiment, the composition provides a therapeutic effect which is an enhanced therapeutic effect compared with that of a composition comprising the same cannabinoids amounts and a smaller amount of the at least one primary terpene, e.g. one half, one third, one quarter or one fifth of that amount. According to various embodiment, the increased therapeutic effect has various forms, e.g. a shortened onset time of the therapeutic effect, an increased magnitude of the therapeutic effect, an extended duration of the therapeutic effect, a reduced requirement for the composition, a reduction in at least one secondary adverse symptom, a reduced frequency of at least one of said conditions and/or symptoms, a reduced severity of at least one of said conditions and/or symptoms, a reduced consumption of other drugs and combinations thereof. According to an embodiment, the increased therapeutic effect comprises a shorter onset time, or differently put, an earlier effect, which is important particularly in cases of sublingual and topical delivery and in cases where a rapid effect is desired, as in treating trauma patients. According to an embodiment, the increased therapeutic effect comprises extended duration of the therapeutic effect, for example an extended time of pain relief. According to an embodiment, the increased therapeutic effect comprises increased magnitude of the therapeutic effect, enabling achieving a desired therapeutic effect on administering smaller doses of cannabinoids, saving thereby on cost. According to an embodiment, the increased therapeutic effect comprises using smaller doses of cannabinoids and still achieving at least the same beneficial result. According to an embodiment, the increased therapeutic effect comprises reduction of secondary adverse symptoms, e.g. adverse symptoms of the main condition, of another condition and/or conditions related to administration of the composition or other drugs. According to an embodiment, the increased therapeutic effect comprises reduced frequency of the conditions and/or symptoms. According to an embodiment, the increased therapeutic effect comprises reduced severity of the conditions and/or symptoms.

According to an embodiment, the composition comprises less than 5% by weight glycol.

According to an embodiment, the composition comprises less than 20% by weight water.

According to an embodiment, the onset time of the therapeutic effect, as measured by methods known in the art, is a time at least 20% shorter than the onset time obtained by administering a composition comprising the same cannabinoids amounts and one fifth the amount of the at least one primary terpene, in some embodiments at least 30%, at least 40%, at least 50% or at least 60% shorter. According to an embodiment, wherein the composition comprises at least two secondary terpenes, the onset time of the therapeutic effect is at least 20% shorter than that of a composition comprising the same cannabinoids amounts, same secondary terpene amounts and one fifth the amount of the at least one primary terpene, at least 30%, at least 40%, at least 50% or at least 60% shorter. Shorter onset time, or differently put an earlier effect, is important particularly in cases of sublingual and topical delivery and in cases where a rapid effect is desired, as in treating pain.

According to an embodiment, the composition provides an onset time of the therapeutic effect which is at least 20% longer than that obtained by administering a composition comprising the same cannabinoids amounts and one fifth the amount of the at least one primary terpene, in some embodiments at least 30%, at least 40%, at least 50% or at least 60% longer. According to an embodiment, wherein the composition comprises at least two secondary terpenes, the onset time of the therapeutic effect is at least 20% longer than that obtained by administering a composition comprising the same cannabinoids amounts, same secondary terpene amounts and one fifth the amount of the at least one primary terpene, in some embodiments at least 30%, at least 40%, at least 50% or at least 60% longer. According to an embodiment, compositions providing delayed onset time are used in combination with shorter onset time to reach a sustained release effect.

According to an embodiment, the magnitude of the therapeutic effect, as measured by methods known in the art, is at least 20% greater than that obtained by administering a composition comprising the same cannabinoids amounts and one fifth the amount of the at least one primary terpene, in some embodiments at least 30%, at least 40%, at least 50% or at least 60% greater. According to an embodiment, wherein the composition comprises at least two secondary terpenes, and the magnitude of the therapeutic effect is at least 20% greater than that obtained by administering a composition comprising the same cannabinoids amounts, same secondary terpene amounts and one fifth the amount of the at least one primary terpene, in some embodiments at least 30%, at least 40%, at least 50% or at least 60% greater. Without wishing to be limited by any particular theory, such increased magnitude of the therapeutic effect may indicate increased bioavailability. Such increased magnitude enables achieving a desired therapeutic effect on administering smaller doses of cannabinoids, saving thereby on cost.

According to an embodiment, the duration of the therapeutic effect, as measured by methods known in the art, is at least 20% longer than that obtained by administering a composition comprising the same cannabinoids amounts and one fifth the amount of the at least one primary terpene, in some embodiments at least 30%, at least 40%, at least 50% at least 60%. at least 70%, at least 80%, at least 90% or at least 100% longer. According to an embodiment, wherein the composition comprises at least two secondary terpenes, the duration of the therapeutic effect is at least 20% longer than that obtained by administering a composition comprising the same cannabinoids amounts, same secondary terpene amounts and one fifth the amount of the at least one primary terpene, in some embodiments at least 30%, at least 40%, at least 50%, at least 60%. at least 70%, at least 80%, at least 90% or at least 100% longer.

According to an embodiment, the frequency of the conditions and/or symptoms, as measured by methods known in the art, is at least 20% smaller compared with that obtained by administration of a composition comprising the same cannabinoids amounts and one fifth the amount of the at least one primary terpene, at least 30%, at least 40%, at least 50% at least 60%. at least 70%, at least 80%, or at least 90% smaller. According to an embodiment, wherein the composition comprises at least two secondary terpenes, the frequency of the conditions and/or symptoms, as measured by methods known in the art, is at least 20% smaller than that obtained by administering a composition comprising the same cannabinoids amounts, same secondary terpene amounts and one fifth the amount of the at least one primary terpene, in some embodiments at least 30%, at least 40%, at least 50%, at least 60%. at least 70%, at least 80%, at least 90% or at least 100% longer.

According to an embodiment, the severity of the conditions and/or symptoms, as measured by methods known in the art, is at least 20% smaller than that obtained by administration of a composition comprising the same cannabinoids amounts and one fifth the amount of the at least one primary terpene, at least 30%, at least 40%, at least 50% at least 60%. at least 70%, at least 80%, or at least 90% smaller. According to an embodiment, wherein the composition comprises at least two secondary terpenes, the severity of the conditions and/or symptoms, as measured by methods known in the art, is at least 20% smaller than that of a composition comprising the same cannabinoids amounts, same secondary terpene amounts and one fifth the amount of the at least one primary terpene, in some embodiments at least 30%, at least 40%, at least 50%, at least 60%. at least 70%, at least 80%, at least 90% or at least 100% longer.

According to an embodiment, the composition is for administration in a vaporizer. According to an embodiment, the composition is for sublingual administration. According to an embodiment, the composition is for topical administration. According to an embodiment, the composition is for administration as a suppository (vaginal or rectal). According to an embodiment, the composition is for administration as a mouthwash. According to an embodiment, the composition is for administration as a varnish.

According to an embodiment, the at least one cannabinoid is selected from the group consisting of CBD, THC, CBC, CBG, CBN, CBL, THCV, CBDV and combinations thereof, the at least one primary terpene is selected from the group consisting of pinene, limonene, caryophyllene, caryophyllene oxide, nerolidol, eucalyptol, terpinene, terpineol, myrcene, cycloartenol, amyrin, camphene, cymene, ocimene, humulene, farnesene, guaiol, eudesmol, friedelin, linalool, camphor, menthol, thymol, carvacrol, eugenol, farnesol, citronellol, geraniol, citral and combinations thereof and the conditions and/or symptoms comprise a fungal infection. According to an embodiment, the fungal infection comprises a dermatophyte infection. According to an embodiment, the dermatophyte infection comprises athlete's foot (Tinea pedis). According to an embodiment, the dermatophyte infection comprises jock itch (Tinea cruris). According to an embodiment, the dermatophyte infection comprises ringworm (Tinea Corporis). According to an embodiment, the dermatophyte infection comprises Tinea Capitis. According to an embodiment, the dermatophyte infection comprises Tinea Cruris. According to an embodiment, the fungal infection comprises Onychomycosis. According to an embodiment, the conditions and/or symptoms comprise Tinea Versicolor. According to an embodiment, the conditions and/or symptoms comprise a yeast infection. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBL. According to an embodiment, the composition comprises THCV. According to an embodiment, the composition comprises CBDV. According to an embodiment, the combinations comprise at least 2 primary terpenes, at least 3, at least 4 or at least 5 primary terpenes. According to an embodiment, the at least one primary terpene is selected from the group consisting of limonene, pinene, caryophyllene, caryophyllene oxide, nerolidol, eucalyptol and combinations thereof. According to an embodiment, the at least one primary terpene is selected from the group consisting of limonene, pinene, caryophyllene, caryophyllene oxide, eucalyptol and combinations thereof. According to an embodiment, the at least one primary terpene is selected from the group consisting of thymol, carvacrol, eugenol, farnesol, citronellol, geraniol, citral and combinations thereof. According to an embodiment, the at least one primary terpene is selected from the group consisting of thymol, carvacrol, eugenol, and combinations thereof. According to an embodiment, the at least one primary terpene is selected from the group consisting of linalool, limonene, caryophyllene and combinations thereof. According to various embodiments at least one primary terpene is selected from the group consisting of pinene, limonene, caryophyllene, caryophyllene oxide, nerolidol, eucalyptol, terpinene, terpineol, myrcene, cycloartenol, amyrin, camphene, cymene, ocimene, humulene, farnesene, guaiol, eudesmol, friedelin, linalool, camphor, menthol, thymol, carvacrol, eugenol, farnesol, citronellol, geraniol and citral and combinations thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral and combinations thereof and the conditions and/or symptoms comprise a Candida infection. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBL. According to an embodiment, the composition comprises THCV. According to an embodiment, the composition comprises CBDV. According to an embodiment, the combinations comprise at least 2 of the terpenes, at least 3, at least 4 or at least 5 of the terpenes. According to an embodiment, the at least one primary terpene is selected from the group consisting of limonene, pinene, caryophyllene, caryophyllene oxide, nerolidol, eucalyptol and combinations thereof. According to an embodiment, the at least one primary terpene is selected from the group consisting of limonene, pinene, nerolidol, caryophyllene, caryophyllene oxide, and combinations thereof. According to an embodiment, the at least one primary terpene is selected from the group consisting of thymol, carvacrol, eugenol, farnesol, citronellol, geraniol, citral and combinations thereof. According to an embodiment, the at least one primary terpene is selected from the group consisting of thymol, carvacrol, eugenol, and combinations thereof. According to an embodiment, the at least one primary terpene is selected from the group consisting of linalool, limonene, caryophyllene and combinations thereof. According to various embodiments at least one primary terpene is selected from the group consisting of pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol and citral and combinations thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of sabine, eucalyptol, pinene, borneol, caryophyllene, geraniol, limonene, myrcene, cycloartenol, citronellol, humulene, linalool, eudesmol, camphor, menthol and combinations thereof and the conditions and/or symptoms comprise a Viral infection. According to an embodiment, the viral infection comprises Molluscum contagiosum. According to an embodiment, the viral infection comprises Viral Warts. According to an embodiment, the viral infection comprises Chickenpox. According to an embodiment, the viral infection comprises Measles. According to an embodiment, the viral infection comprises Rubella. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBL. According to an embodiment, the composition comprises THCV. According to an embodiment, the composition comprises CBDV. According to an embodiment, the combinations comprise at least 2 of the terpenes, at least 3, at least 4 or at least 5 of the terpenes. According to an embodiment, the at least one primary terpene is selected from the group consisting of eucalyptol, caryophyllene, pinene, borneol, linalool and combination thereof. According to various embodiments at least one primary terpene is selected from the group consisting of sabine, eucalyptol, pinene, borneol, caryophyllene, geraniol, limonene, myrcene, cycloartenol, citronellol, humulene, linalool, eudesmol, camphor and menthol and combinations thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of geraniol, eucalyptol, pinene, borneol, caryophyllene, terpineol, limonene, myrcene, cycloartenol, citronellol, humulene, linalool, eudesmol, camphor, menthol and combinations thereof and the conditions and/or symptoms comprise Herpes, optionally Herpes simplex, and/or Herpes Zoster. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of geraniol, eucalyptol, caryophyllene, pinene, terpineol, borneol, linalool, limonene and combination thereof. According to an embodiment, the at least one primary terpene is selected from the group consisting of geraniol, caryophyllene, pinene, terpineol, limonene and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of eucalyptol, citronellol, pinene, borneol, caryophyllene, geraniol, limonene, myrcene, cycloartenol, citronellol, humulene, linalool, eudesmol, camphor, menthol and combinations thereof and the conditions and/or symptoms comprise Hand Foot and Mouth disease. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of eucalyptol, caryophyllene, pinene, borneol, linalool and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, fenchol, geranyl acetate, camphor, menthol and combinations thereof and the conditions and/or symptoms comprise Bacterial infection. According to an embodiment, the conditions and/or symptoms comprise Necrotizing Fasciitis. According to an embodiment, the conditions and/or symptoms comprise Staphylococcal Scalded Skin Syndrome (SSSS). According to an embodiment, the conditions and/or symptoms comprise Leprosy. According to an embodiment, the conditions and/or symptoms comprise Cellulitis. According to an embodiment, the conditions and/or symptoms comprise Erysipelas. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of pinene, limonene, eucalyptol, caryophyllene, amyrin, linalool and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, camphor, menthol and combinations thereof and the conditions and/or symptoms comprise acne. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of limonene, linalool, pinene, terpineol, caryophyllene and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, camphor, menthol and combinations thereof and the conditions and/or symptoms comprise Methycillin-Resistant Staphylococcus Aureus (MRSA). According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of pinene, limonene, amyrin, eucalyptol, linalool and combination thereof.

According to an embodiment, the at least one cannabinoid is selected from the group consisting of CBD, THC, CBC, CBG, CBN, CBDa and THCa, CBLA, THCV and combinations thereof, the at least one primary terpene is selected from the group consisting of pinene, limonene, linalool, germacrene, caryophyllene, nerolidol, terpineol, terpinene, eucalyptol, myrcene, cirtonellol, humulene, eudesmol, camphor, menthol and combinations thereof and the conditions and/or symptoms comprise ulcer, optionally pressure ulcer and/or diabetic ulcer. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of pinene, limonene, linalool, germacrene, caryophyllene, eucalyptol and combination thereof.

According to an embodiment, the at least one cannabinoid is selected from the group consisting of CBD, THC, CBC, CBG, CBN, CBDa and THCa, CBLA, THCV and combinations thereof, the at least one primary terpene is selected from the group consisting of pinene, limonene, linalool, germacrene, caryophyllene, nerolidol, terpineol, terpinene, eucalyptol, myrcene, cirtonellol, humulene, eudesmol, camphor, menthol and combinations thereof and the conditions and/or symptoms comprise oral ulcer and/or mucositis. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of pinene, limonene, linalool, caryophyllene, eucalyptol and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of camphene, sabinene, eugenol, cirtonellol, gerniol, terpineol, pinene, limonene, linalool, germacrene, caryophyllene, nerolidol, terpinene, eucalyptol, myrcene, humulene, eudesmol, camphor, menthol and combinations thereof and the conditions and/or symptoms are selected from the group consisting of dermatitis, eczema, and seborrhea and combinations thereof. According to an embodiment, the conditions and/or symptoms comprise Lichen planus. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of linalool, camphene, sabinene, limonene, myrcene.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, nerolidol, Cycloartenol, caryophyllene, caryophyllene oxide, amyrin, eucalyptol, myrcene, cirtonellol, humulene, linalool, farnesene, phellandrene, Cymenen, eudesmol, carvacrol, eugenol, camphor, menthol and combinations thereof and conditions and/or symptoms comprise a periodontal disease. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of carvacrol, eugenol, pinene, eucalyptol, amyrin and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of linalool, nerolidol, pinene, limonene, germacrene, caryophyllene, terpineol, terpinene, eucalyptol, myrcene, cirtonellol, humulene, eudesmol, camphor, menthol and combinations thereof and the conditions and/or symptoms comprise Leishmaniasis. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of linalool, nerolidol, pinene, caryophyllene, eucalyptol and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of caryophyllene, nerolidol, pinene, eucalyptol, limonene, myrcene, cirtonellol, humulene, terpineol, terpinene, guaiol, friedelin, eudesmol, linalool, camphor, menthol and combinations thereof and the conditions and/or symptoms comprise malaria. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of caryophyllene, nerolidol, pinene, eucalyptol, limonene and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of caryophyllene, nerolidol, pinene, eucalyptol, limonene, myrcene, cirtonellol, humulene, terpineol, terpinene, guaiol, friedelin, eudesmol, linalool, camphor, menthol and combinations thereof and the conditions and/or symptoms comprise scabies. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of camphene, linalool, sabinene, limonene and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of myrcene, linalool, caryophyllene, eucalyptol, borneol, pinene, sabinene, terpineol, camphor, menthol and combinations thereof the conditions and/or symptoms are selected from the group consisting of local pain, local itch and/or local burn. According to an embodiment, the therapeutic effect treats Temporomandibular-Joint (TMJ). According to an embodiment, the composition comprises CBD. According to an embodiment, the composition comprises THC. According to an embodiment, the composition comprises CBC. According to an embodiment, the composition comprises CBN. According to an embodiment, the combinations comprise at least 2 of the terpenes, at least 3, at least 4 or at least 5 of the terpenes. According to an embodiment, the at least one primary terpene is selected from the group consisting of linalool, myrcene, borneol, pinene, eucalyptol and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, eucalyptol, caryophyllene myrcene, citronellol, humulene, terpinene, terpineol, guaiol, friedelin, eudesmol linalool, borneol, amyrin, cycolartenol, camphor, menthol and combinations thereof and the conditions and/or symptoms are selected from the group consisting of an inflammatory injury, wound, sore and ulcer or combinations thereof. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of myrcene, humulene, linalool, caryophyllene, eucalyptol and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of linalool, caryophyllene, limonene, eucalyptol, myrcene, cirtonellol, pinene, sabinene, terpineol, terpinene, camphor, menthol and combinations thereof and the conditions and/or symptoms comprise Psoriasis. According to an embodiment, the composition comprises CBD. According to an embodiment, the composition comprises THC. According to an embodiment, the composition comprises CBG. According to an embodiment, the composition comprises CBN. According to an embodiment, the composition comprises CBDa. According to an embodiment, the composition comprises THCa. According to an embodiment, the combinations comprise at least 2 of the terpenes, at least 3, at least 4 or at least 5 of the terpenes. According to an embodiment, the at least one primary terpene is selected from the group consisting of linalool, caryophyllene, limonene, sabinene, terpinene and combination thereof.

According to an embodiment, the at least one cannabinoid is selected from the group consisting of CBD, THC and CBN, and combinations thereof, the at least one primary terpene is selected from the group consisting of terpinolene, linalool, amyrin, pinene, borneol, caryophyllene, geraniol and combinations thereof and the therapeutic effect provides an increased immune response. According to an embodiment, the composition comprises CBD. According to an embodiment, the composition comprises THC. According to an embodiment, the composition comprises CBN. According to an embodiment, the combinations comprise at least 2 of the terpenes, at least 3, at least 4 or at least 5 of the terpenes.

According to an embodiment, the composition comprises at least one of CBD, THC and CBN, and combinations thereof, the at least one primary terpene is selected from the group consisting of terpinolene, linalool, amyrin, caryophyllene and combinations thereof and the therapeutic effect provides an increased immune response. According to an embodiment, the composition comprises CBD. According to an embodiment, the composition comprises THC. According to an embodiment, the composition comprises CBN. According to an embodiment, the combinations comprise at least 2 of the terpenes, at least 3, or all 4 of the terpenes.

According to an embodiment, the terpene-enriched cannabinoid composition is for administration to a human subject. According to an embodiment, the terpene-enriched cannabinoid composition is for use as a veterinary medication.

According to an embodiment, the shelf life of the composition is at least 6 months or at least a year. According to an embodiment, primary terpene degradation in the composition is less than 20% per year.

According to an embodiment, further provided is a product comprising tablets, gel capsules, suppositories, energy drinks, bakery products, medical patches, mouthwashes, varnishes, cigarettes and vaporizer liquids containing the composition.

According to an embodiment, further provided is a commercial product comprising two compositions according to the present invention, which two compositions differ in the content of the at least one primary terpene. According to an embodiment, further provided is a commercial product comprising two compositions according to the present invention, which two compositions comprise different primary terpenes.

According to an embodiment, further provided is a method for treating conditions and/or symptoms selected from the group consisting of ulcer, viral infection, bacterial infection, fungal infection, infectious disease, and inflammation and combinations thereof, the method comprising administering to a subject in need thereof a therapeutically effective amount of a composition comprising (i) at least one cannabinoid in a specific amount, (ii) at least one primary terpene in a specific amount and (iii) optionally at least two secondary terpenes.

According to an embodiment, further provided is a composition comprising (i) at least one cannabinoid in a specific amount, (ii) at least one primary terpene in a specific amount and (iii) optionally at least two secondary terpenes, for use in the treatment of conditions and/or symptoms selected from the group consisting of ulcer, viral infection, bacterial infection, fungal infection, infectious disease, and inflammation and combinations thereof.

According to an embodiment, further provided is the use of a composition comprising (i) at least one cannabinoid in a specific amount, (ii) at least one primary terpene in a specific amount and (iii) optionally at least two secondary terpenes, in the preparation of a medicament for the treatment of conditions and/or symptoms selected from the group consisting of ulcer, viral infection, bacterial infection, fungal infection, infectious disease, and inflammation and combinations thereof.

According to an embodiment, the composition is for administration in a form selected from the group consisting of cigarettes, vaporizer plant material, vaporizer liquid, extract, tablets, gel capsules, suppositories, mouthwashes, varnishes and combinations thereof. According to an embodiment, the daily dose of the composition comprises about 1 milligram cannabinoid to about 300 milligram, about 2 milligram cannabinoid to about 200 milligram or about 3 milligram cannabinoid to about 100 milligram. According to another embodiment, the daily dose of the composition comprises about 0.5 milligram cannabinoid per kilogram body weight to about 30 milligram cannabinoid per kilogram body weight.

According to an embodiment, further provided is a method for treating conditions and/or symptoms selected from the group consisting of ulcer, viral infection, bacterial infection, fungal infection and inflammation and combinations thereof, the method comprising administering to a subject in need thereof a therapeutically effective amount of a composition comprising (i) at least one cannabinoid in a specific amount, (ii) at least one primary terpene in a specific amount and (iii) optionally at least two secondary terpenes.

According to an embodiment, further provided is a composition comprising (i) at least one cannabinoid in a specific amount, (ii) at least one primary terpene in a specific amount and (iii) optionally at least two secondary terpenes for use in the treatment of conditions and/or symptoms selected from the group consisting of ulcer, viral infection, bacterial infection, fungal infection and inflammation and combinations thereof.

According to an embodiment, further provided is the use of a composition comprising (i) at least one cannabinoid in a specific amount, (ii) at least one primary terpene in a specific amount and (iii) optionally at least two secondary terpenes in the preparation of a medicament for the treatment of conditions and/or symptoms selected from the group consisting of ulcer, viral infection, bacterial infection, fungal infection and inflammation and combinations thereof.

According to an embodiment, the composition is administered in a form selected from the group consisting of cigarettes, vaporizer plant material, vaporizer liquid, extract, tablets, gel capsules, suppositories, mouthwashes, varnishes and combinations thereof. According to an embodiment, the daily dose of the composition comprises about 1 milligram cannabinoid to about 300 milligram, about 2 milligram cannabinoid to about 200 milligram or about 3 milligram cannabinoid to about 100 milligram. According to another embodiment, the daily dose of the composition comprises about 0.5 milligram cannabinoid per kilogram body weight to about 30 milligram cannabinoid per kilogram body weight.

According to an embodiment, the conditions and/or symptoms are associated with at least one of skin ulcer, skin viral infection, skin bacterial infection, skin fungal infection, skin infectious disease and skin inflammation.

According to an embodiment, the composition is administered in a vaporizer. According to an embodiment, the composition is administered sublingually. According to an embodiment, the composition is administered topically. According to an embodiment, the composition is administered as a suppository (vaginal or rectal). According to an embodiment, the composition is administered as a mouthwash. According to an embodiment, the composition is administered as a varnish.

According to an embodiment, the composition comprises less than 5% by weight glycol. According to an embodiment, the composition comprises less than 20% by weight water. According to an embodiment, the composition comprises at least 5% by weight of a non-cannabinoid, non-terpene carrier. According to an embodiment, the non-cannabinoid, non-terpene carrier comprises cellulose and a weight/weight ratio of the total terpenes to total cannabinoids in the composition is from about 0.1:1 to about 1:1, According to an embodiment, the non-cannabinoid, non-terpene carrier comprises less than 5% by weight cellulose and a weight/weight ratio of the total terpenes to total cannabinoids in the composition is from about 0.05:1 to about 1:1.

According to an embodiment, the conditions and/or symptoms are selected from the group consisting of fungal infection, dermatophytes, candida infection, athlete's foot (Tinea pedis), yeast infection, jock itch (Tinea cruris), ringworm (Tinea Corporis), Tinea Capitis, Onychomycosis, Tinea Versicolor, Tinea Cruris, viral infection, Herpes, Herpes simplex, Herpes Zoster, Hand Foot and Mouth disease, Molluscum contagiosum, Viral Warts, Chickenpox, Measles, Rubella, bacterial infection, acne, Methycillin-Resistant Staphylococcus Aureus (MRSA), Necrotizing Fasciitis, Staphylococcal Scalded Skin Syndrome (SSSS), Leprosy, Cellulitis, Erysipelas, ulcer, pressure ulcer, diabetic ulcer, oral ulcer, mucositis, dermatitis, eczema, seborrhea, Lichen planus, periodontal disease, leishmaniasis, scabies, an inflammatory injury, psoriasis, local pain, local itch and/or local burn, Temporomandibular-Joint (TMJ) and combination thereof.

According to an embodiment, the therapeutic effect comprises an increased immune response.

According to an embodiment, the composition further comprises an additive selected from the group consisting of antioxidants, emulsifiers and texturizers vegetable oils, plant extracts, honey, sucrose, glucose and fructose, pharmaceutical excipients and combinations thereof.

According to an embodiment the composition comprises tetrahydrocannabinol (THC) and/or tetrahydrocannabinolic acid (THCa), wherein THC and/or THCa is in a total concentration of at least 1% by weight, at least 2%, at least 3%, at least 4%, at least 5%, at least 6%, at least 8%, at least 10%, at least 12%, at least 14%, at least 16%, at least 18%, or at least 20%, at least 30%, at least 40%, at least 50%, at least 60%, at least 70%, at least 80%, or at least 90% by weight.

According to an embodiment, the composition comprises cannabidiol (CBD) and/or cannabidiolic acid (CBDa), wherein CBD and/or CBDa is in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the composition comprises tetrahydrocannabinol (THC) and/or tetrahydrocannabinolic acid (THCa) and cannabidiol (CBD) and/or cannabidiolic acid (CBDa), wherein, THC and/or THCa is in a total concentration of at least 2.5% by weight, and CBD and/or CBDa is in a total concentration of at least 2.5% by weight; THC and/or THCa in a total concentration of at least 3% by weight, and CBD and/or CBDa in a total concentration of at least 3% by weight; THC and/or THCa in a total concentration of at least 4% by weight, and CBD and/or CBDa in a total concentration of at least 4% by weight; THC and/or THCa in a total concentration of at least 5% by weight, and CBD and/or CBDa in a total concentration of at least 5% by weight; THC and/or THCa in a total concentration of at least 8% by weight, and CBD and/or CBDa in a total concentration of at least 8% by weight; THC and/or THCa in a total concentration of at least 10% by weight, and c CBD and/or CBDa in a total concentration of at least 10% by weight.

According to a related embodiment, the composition comprises cannabigerol (CBG) and/or cannabigerol acid (CBGa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises cannabinol (CBN) and/or cannabinol acid (CBNa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises cannabichromene (CBC) and/or cannabichromenic acid (CBCa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises cannabicyclol (CBL) and/or cannabicyclol acid (CBLa) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises tetrahydrocannabivarin (THCV), and/or tetrahydrocannabivarin acid (THCVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises cannabidivarin (CBDV) and/or cannabigerovarin acid (CBGVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight is a vaporizer cannabis filling material or is a cannabis cigarette.

According to an embodiment, the administered composition comprises (a) at least one cannabinoid from the group consisting of THC, CBD, CBG, CBC, CBN, CBL and non-decarboxylated form thereof; (b) less than 5% by weight cellulose; (c) primary terpene forming at least 40% by weight of the total terpene content, and (d) primary terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, geranyl acetate, camphor, menthol, iso-menthone, neral, gerial, viridiflorol, germacrene, thymol, Menth-2-en-1-ol, farensol, carotol, myrtenol and combinations thereof. According to an embodiment, the composition comprises less than 5% wt cellulose and THC and/or THCa and/or CBD and/or CBDa in a concentration of at least 5% by weight, at least 10%, at least 15%, at least 20%, at least 25%, at least 30%, at least 35%, at least 40%, at least 45% or at least 50% by weight. According to an embodiment, the composition comprises less than 5% wt cellulose and CBG and/or CBGa and/or CBC and/or CBCa and/or CBN and/or CBNa in a concentration of at least 0.5% by weight, at least 0.75%, at least 1%, at least 1.25%, at least 1.5%, at least 1.75%, at least 2%, at least 2.25%, at least 2.5% or at least 3% by weight. According to an embodiment, the composition comprises less than 5% wt cellulose and water in a concentration of less than 5% by weight, less than 4%, less than 3%, less than 2%, or less than 1% by weight. According to an embodiment, the composition comprises less than 5% by weight cellulose, and the primary terpenes forms at least 40% by weight of the total terpene content, at least 50%, at least 60%, at least 70%, at least 80% or at least 90% by weight and the at least one primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, geranyl acetate, camphor, menthol, iso-menthone, neral, gerial, viridiflorol, germacrene, thymol, Menth-2-en-1-ol, farensol, carotol, myrtenol and combinations thereof.

According to an embodiment, the composition is liquid. According to an embodiment, the composition is selected from the group consisting of vaporizer cannabis filling liquid, cannabis tablets, cannabis suppositories, cannabis gel capsules, cannabis candies, cannabis drinks, cannabis mouthwashes, cannabis varnishes and cannabis baked products.

According to an embodiment, the at least one primary terpene of the administered composition is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, geranyl acetate, camphor, menthol, iso-menthone, neral, gerial, viridiflorol, germacrene, thymol, Menth-2-en-1-ol, farensol, carotol, myrtenol and combinations thereof.

According to an embodiment, the composition comprises less than 5% by weight glycol.

According to an embodiment, the composition comprises less than 20% by weight water.

According to an embodiment, the composition provides a therapeutic effect which is an enhanced therapeutic effect compared with that of a composition comprising the same cannabinoids amounts and one fifth the amount of the at least one primary terpene.

According to an embodiment, the enhanced therapeutic effect is selected from the group consisting of a shortened onset time of said therapeutic effect, an increased magnitude of said therapeutic effect, an extended duration of said therapeutic effect, a reduced requirement for the composition, a reduction in at least one secondary adverse symptom, a reduced frequency of at least one of said conditions and/or symptoms, a reduced severity of said conditions and/or symptoms, a reduced consumption of other drugs and combinations thereof.

According to an embodiment, the at least one primary terpene and the cannabinoids are present in specific amounts, and the shortened onset time of the therapeutic effect is a time at least 20% shorter than of the onset time obtained by administering a composition comprising the same cannabinoids amounts and one fifth the amount of the at least one primary terpene, in some embodiments at least 30%, at least 40%, at least 50% or at least 60% shorter.

According to an embodiment, the increased magnitude of the therapeutic effect is a magnitude of at least 20% greater than the magnitude obtained by administering a composition comprising the same cannabinoids amounts and one fifth the amount of the at least one primary terpene, in some embodiments at least 30%, at least 40%, at least 50% at least 60%, at least 70%, at least 80%, at least 90% or at least 100% greater.

According to an embodiment, the extended duration of the therapeutic effect is a duration of at least 20% longer than the duration obtained by administering a composition comprising the same cannabinoids amounts and one fifth the amount of the at least one primary terpene, in some embodiments at least 30%, at least 40%, at least 50% at least 60%, at least 70%, at least 80%, at least 90% or at least 100% greater.

According to an embodiment, the reduced requirement for said composition is a reduction of at least 20% in the dosage required to obtain the same therapeutic effect as that obtained by administering a dosage of a composition comprising the same cannabinoids amounts and one fifth the amount of the at least one primary terpene, in some embodiments a reduction of at least 30%, at least 40%, at least 50% at least 60%, at least 70%, at least 80%, at least 90% or at least 100%.

According to an embodiment, a reduction in at least one secondary adverse symptom is a decrease of at least 20% in a frequency, extent or severity of the adverse symptoms compared with a frequency, extent or severity of the adverse symptoms obtained by administering a composition comprising the same cannabinoids amounts and one fifth the amount of the at least one primary terpene, in some embodiments a decrease of at least 30%, at least 40%, at least 50% at least 60%, at least 70%, at least 80%, at least 90% or at least 100%.

According to an embodiment, the reduced frequency of the conditions and/or symptoms is a reduction of at least 20% compared with of the frequency of the conditions and/or symptoms obtained by administering a composition comprising the same cannabinoids amounts and one fifth the amount of the at least one primary terpene, in some embodiments a reduction of at least 30%, at least 40%, at least 50% at least 60%, at least 70%, at least 80%, or at least 900%.

According to an embodiment, the reduced severity of the conditions and/or symptoms is a reduction of at least 20% compared with a severity of the conditions and/or symptoms obtained by administering a composition comprising the same cannabinoids amounts and one fifth the amount of the at least one primary terpene, in some embodiments a reduction of at least 30%, at least 40%, at least 50% at least 60%, at least 70%, at least 80%, or at least 900%.

According to an embodiment, the reduced consumption of other drugs is a reduction of at least 20% in the amount or frequency of other drugs consumed in order to obtain the same therapeutic effect compared with that obtained by administering a composition comprising the same cannabinoids amounts and one fifth the amount of the at least one primary terpene, in some embodiments a reduction of at least 30%, at least 40%, at least 50% at least 60%, at least 70%, at least 80%, or at least 90%.

According to an embodiment, the at least one of at least one cannabinoid is selected from the group consisting of CBD, THC, CBC, CBG, CBN, CBL, CBDV, THCV and combinations thereof, the at least one primary terpene is selected from the group consisting of pinene, limonene, caryophyllene, caryophyllene oxide, nerolidol, eucalyptol, terpinene, terpineol, myrcene, cycloartenol, amyrin, camphene, cymene, ocimene, humulene, farnesene, guaiol, eudesmol, friedelin, linalool, camphor, menthol, thymol, carvacrol, eugenol, farnesol, citronellol, geraniol, citral and combinations thereof and the conditions and/or symptoms comprise a fungal infection. According to an embodiment, the conditions and/or symptoms comprise dermatophytes. According to an embodiment, the conditions and/or symptoms comprise athlete's foot (Tinea pedis). According to an embodiment, the conditions and/or symptoms comprise a yeast infection. According to an embodiment, the conditions and/or symptoms comprise jock itch (Tinea cruris). According to an embodiment, the conditions and/or symptoms comprise ringworm (Tinea Corporis). According to an embodiment, the conditions and/or symptoms comprise Tinea Capitis. According to an embodiment, the conditions and/or symptoms comprise Onychomycosis. According to an embodiment, the conditions and/or symptoms comprise Tinea Versicolor. According to an embodiment, the conditions and/or symptoms comprise Tinea Cruris. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBL. According to an embodiment, the composition comprises THCV. According to an embodiment, the composition comprises CBDV. According to an embodiment, the combinations comprise at least 2 of the terpenes, at least 3, at least 4 or at least 5 of the terpenes. According to an embodiment, the at least one primary terpene is selected from the group consisting of limonene, pinene, caryophyllene, caryophyllene oxide, nerolidol, eucalyptol and combinations thereof. According to an embodiment, the at least one primary terpene is selected from the group consisting of limonene, pinene, caryophyllene, caryophyllene oxide, eucalyptol and combinations thereof. According to an embodiment, the at least one primary terpene is selected from the group consisting of thymol, carvacrol, eugenol, farnesol, citronellol, geraniol, citral and combinations thereof. According to an embodiment, the at least one primary terpene is selected from the group consisting of thymol, carvacrol, eugenol, and combinations thereof. According to an embodiment, the at least one primary terpene is selected from the group consisting of linalool, limonene, caryophyllene and combinations thereof. According to various embodiments at least one primary terpene comprises pinene, limonene, caryophyllene, caryophyllene oxide, nerolidol, eucalyptol, terpinene, terpineol, myrcene, cycloartenol, amyrin, camphene, cymene, ocimene, humulene, farnesene, guaiol, eudesmol, friedelin, linalool, camphor, menthol, thymol, carvacrol, eugenol, farnesol, citronellol, geraniol and citral and combinations thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral and combinations thereof and the conditions and/or symptoms comprise a Candida infection. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBL. According to an embodiment, the composition comprises THCV. According to an embodiment, the composition comprises CBDV. According to an embodiment, the combinations comprise at least 2 of the terpenes, at least 3, at least 4 or at least 5 of the terpenes. According to an embodiment, the at least one primary terpene is selected from the group consisting of limonene, pinene caryophyllene, caryophyllene oxide, nerolidol, eucalyptol and combinations thereof. According to an embodiment, the least one primary terpene is selected from the group consisting of limonene, pinene, nerolidol, caryophyllene, caryophyllene oxide, and combinations thereof. According to an embodiment, the at least one primary terpene is selected from the group consisting of thymol, carvacrol, eugenol, farnesol, citronellol, geraniol, citral and combinations thereof. According to an embodiment, the at least one primary terpene is selected from the group consisting of thymol, carvacrol, eugenol, and combinations thereof. According to an embodiment, the at least one primary terpene is selected from the group consisting of linalool, limonene, caryophyllene and combinations thereof. According to various embodiments at least one primary terpene comprises pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol and citral and combinations thereof.

According to an embodiment, the at least one cannabinoid is selected from the group consisting of at least one of CBD, THC, CBC, CBG, CBN, CBL, CBDV, THCV and combinations thereof, the at least one primary terpene is selected from the group consisting of sabine, eucalyptol, pinene, borneol, caryophyllene, geraniol, limonene, myrcene, cycloartenol, citronellol, humulene, linalool, eudesmol, camphor, menthol and combinations thereof and the conditions and/or symptoms comprise a Viral infection. According to an embodiment, the conditions and/or symptoms comprise Molluscum contagiosum. According to an embodiment, the conditions and/or symptoms comprise Viral Warts. According to an embodiment, the conditions and/or symptoms comprise Chickenpox. According to an embodiment, the conditions and/or symptoms comprise Measles. According to an embodiment, the conditions and/or symptoms comprise Rubella. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBL. According to an embodiment, the composition comprises THCV. According to an embodiment, the composition comprises CBLA. According to an embodiment, the composition comprises CBDV. According to an embodiment, the combinations comprise at least 2 of the terpenes, at least 3, at least 4 or at least 5 of the terpenes. According to an embodiment, the at least one primary terpene is selected from the group consisting of eucalyptol, caryophyllene, pinene, borneol, linalool and combination thereof. According to various embodiments at least one primary terpene comprises sabine, eucalyptol, pinene, borneol, caryophyllene, geraniol, limonene, myrcene, cycloartenol, citronellol, humulene, linalool, eudesmol, camphor and menthol and combinations thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of geraniol, eucalyptol, pinene, borneol, caryophyllene, terpineol, limonene, myrcene, cycloartenol, citronellol, humulene, linalool, eudesmol, camphor, menthol and combinations thereof and the conditions and/or symptoms comprise Herpes, optionally Herpes simplex, and/or Herpes Zoster. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of geraniol, eucalyptol, caryophyllene, pinene, terpineol, borneol, linalool, limonene and combination thereof. According to an embodiment, the at least one primary terpene is selected from the group consisting of geraniol, caryophyllene, pinene, terpineol, limonene and combination thereof.

According to an embodiment, the at least one cannabinoid is selected from the group consisting of at least one of CBD, THC, CBC, CBG, CBN, CBL, CBDV, THCV and combinations thereof, the at least one primary terpene is selected from the group consisting of eucalyptol, citronellol, pinene, borneol, caryophyllene, geraniol, limonene, myrcene, cycloartenol, citronellol, humulene, linalool, eudesmol, camphor, menthol and combinations thereof and the conditions and/or symptoms comprise Hand Foot and Mouth disease. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of eucalyptol, caryophyllene, pinene, borneol, linalool and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, fenchol, geranyl acetate, camphor, menthol and combinations thereof and the conditions and/or symptoms comprise a Bacterial infection. According to an embodiment, the conditions and/or symptoms comprise Necrotizing Fasciitis. According to an embodiment, the conditions and/or symptoms comprise Staphylococcal Scalded Skin Syndrome (SSSS). According to an embodiment, the conditions and/or symptoms comprise Leprosy. According to an embodiment, the conditions and/or symptoms comprise Cellulitis. According to an embodiment, the conditions and/or symptoms comprise Erysipelas. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of pinene, limonene, eucalyptol, caryophyllene, amyrin, linalool and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, camphor, menthol and combinations thereof and the conditions and/or symptoms comprise acne. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of limonene, linalool, pinene, terpineol, caryophyllene and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, camphor, menthol and combinations thereof and the conditions and/or symptoms comprise Methycillin-Resistant Staphylococcus Aureus (MRSA). According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of pinene, limonene, amyrin, eucalyptol, linalool and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, linalool, germacrene, caryophyllene, nerolidol, terpineol, terpinene, eucalyptol, myrcene, cirtonellol, humulene, eudesmol, camphor, menthol and combinations thereof and the conditions and/or symptoms comprise ulcer, optionally pressure ulcer and/or diabetic ulcer. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of pinene, limonene, linalool, germacrene, caryophyllene, eucalyptol and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, linalool, germacrene, caryophyllene, nerolidol, terpineol, terpinene, eucalyptol, myrcene, cirtonellol, humulene, eudesmol, camphor, menthol and combinations thereof and the conditions and/or symptoms comprise oral ulcer and/or mucositis. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of pinene, limonene, linalool, caryophyllene, eucalyptol and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of camphene, sabinene, eugenol, cirtonellol, gerniol, terpineol, pinene, limonene, linalool, germacrene, caryophyllene, nerolidol, terpinene, eucalyptol, myrcene, humulene, eudesmol, camphor, menthol and combinations thereof and the conditions and/or symptoms are selected from the group consisting of dermatitis, eczema, and seborrhea and combinations thereof. According to an embodiment, the conditions and/or symptoms comprise Lichen planus. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of linalool, camphene, sabinene, limonene, myrcene.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, nerolidol, Cycloartenol, caryophyllene, caryophyllene oxide, amyrin, eucalyptol, myrcene, cirtonellol, humulene, linalool, farnesene, phellandrene, Cymenen, eudesmol, carvacrol, eugenol, camphor, menthol and combinations thereof and the conditions and/or symptoms comprise a periodontal disease. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of carvacrol, eugenol, pinene, eucalyptol, amyrin and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of linalool, nerolidol, pinene, limonene, germacrene, caryophyllene, terpineol, terpinene, eucalyptol, myrcene, cirtonellol, humulene, eudesmol, camphor, menthol and combinations thereof and the conditions and/or symptoms comprise Leishmaniasis. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of linalool, nerolidol, pinene, caryophyllene, eucalyptol and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of caryophyllene, nerolidol, pinene, eucalyptol, limonene, myrcene, cirtonellol, humulene, terpineol, terpinene, guaiol, friedelin, eudesmol, linalool, camphor, menthol and combinations thereof and the conditions and/or symptoms comprise Malaria. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of caryophyllene, nerolidol, pinene, eucalyptol, limonene and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of caryophyllene, nerolidol, pinene, eucalyptol, limonene, myrcene, cirtonellol, humulene, terpineol, terpinene, guaiol, friedelin, eudesmol, linalool, camphor, menthol and combinations thereof and conditions and/or symptoms comprise scabies. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of camphene, linalool, sabinene, limonene and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of myrcene, linalool, caryophyllene, eucalyptol, borneol, pinene, sabinene, terpineol, camphor, menthol and combinations thereof and conditions and/or symptoms are selected from the group consisting of local pain, local itch and/or local burn. According to an embodiment, the therapeutic effect treats Temporomandibular-Joint (TMJ). According to an embodiment, the composition comprises CBD. According to an embodiment, the composition comprises THC. According to an embodiment, the composition comprises CBC. According to an embodiment, the composition comprises CBN. According to an embodiment, the combinations comprise at least 2 of the terpenes, at least 3, at least 4 or at least 5 of the terpenes. According to an embodiment, the at least one primary terpene is selected from the group consisting of linalool, myrcene, borneol, pinene, eucalyptol and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of pinene, limonene, eucalyptol, caryophyllene myrcene, citronellol, humulene, terpinene, terpineol, guaiol, friedelin, eudesmol linalool, borneol, amyrin, cycolartenol, camphor, menthol and combinations thereof and the conditions and/or symptoms are selected from the group consisting of an inflammatory injury, wound, sore and ulcer and combinations thereof. According to an embodiment, the composition comprises CBD. According to an embodiment, the composition comprises THC. According to an embodiment, the composition comprises CBC. According to an embodiment, the composition comprises CBG. According to an embodiment, the composition comprises CBN. According to an embodiment, the composition comprises CBDa. According to an embodiment, the composition comprises THCa. According to an embodiment, the composition comprises CBLA. According to an embodiment, the composition comprises THCV. According to an embodiment, the combinations comprise at least 2 of the terpenes, at least 3, at least 4 or at least 5 of the terpenes. According to an embodiment, the at least one primary terpene is selected from the group consisting of myrcene, humulene, linalool, caryophyllene, eucalyptol and combination thereof.

According to an embodiment, the at least one cannabinoid is selected from the group consisting of CBD, THC, CBC, CBG, CBN, CBL, CBDV, THCV and combinations thereof, the at least one primary terpene is selected from the group consisting of linalool, caryophyllene, limonene, eucalyptol, myrcene, cirtonellol, pinene, sabinene, terpineol, terpinene, camphor, menthol and combinations thereof and the conditions and/or symptoms comprise Psoriasis. According to an embodiment, the composition comprises CBD. According to an embodiment, the composition comprises THC. According to an embodiment, the composition comprises CBG. According to an embodiment, the composition comprises CBN. According to an embodiment, the composition comprises CBDa. According to an embodiment, the composition comprises THCa. According to an embodiment, the combinations comprise at least 2 of the terpenes, at least 3, at least 4 or at least 5 of the terpenes. According to an embodiment, the at least one primary terpene is selected from the group consisting of linalool, caryophyllene, limonene, sabinene, terpinene and combination thereof.

According to an embodiment, the at least one cannabinoid is selected from the group consisting of CBD, THC and CBN and combinations thereof, the at least one primary terpene is selected from the group consisting of terpinolene, linalool, amyrin, pinene, borneol, caryophyllene, geraniol and combinations thereof and the therapeutic effect comprises an increase immune response. According to an embodiment, the composition comprises CBD. According to an embodiment, the composition comprises THC. According to an embodiment, the composition comprises CBN. According to an embodiment, the combinations comprise at least 2 of the terpenes, at least 3, at least 4 or at least 5 of the terpenes

According to an embodiment, the at least one cannabinoid is selected from the group consisting of CBD, THC and CBN and combinations thereof, the at least one primary terpene is selected from the group consisting of terpinolene, linalool, amyrin, caryophyllene and combinations thereof and the therapeutic effect comprises an increased immune response. According to an embodiment, the composition comprises CBD. According to an embodiment, the composition comprises THC. According to an embodiment, the composition comprises CBN. According to an embodiment, the combinations comprise at least 2 of the terpenes, at least 3, or all 4 of the terpenes.

According to an embodiment, the method comprises (i) administering to the patient for a first period of time a first terpene-enriched cannabis composition comprising a first cannabinoid at a first cannabinoid amount and a first primary terpene at a first primary terpene amount, followed by (ii) administering to the subject for a second period of time a second terpene-enriched cannabis composition comprising the first cannabinoid amount at a first cannabinoid amount and a second primary terpenes at a second primary terpene amount. According to an embodiment, the method further comprises administering to the patient for a third period of time the first terpene-enriched cannabis composition comprising the first cannabinoid amount and a third primary terpene at a third primary terpene amount. According to an embodiment, the third primary terpene is identical to the first primary terpene.

According to another embodiment, the method for treating a patient comprises (i) administering to the patient a first terpene-enriched cannabis composition comprising a first cannabinoid at a first cannabinoid amount and a first primary terpene at a first primary terpene amount and administering to the patient, at least 2 hours later a second terpene-enriched cannabis composition comprising the first cannabinoid at a first cannabinoid amount and a second primary terpenes at a second primary terpene amount. According to another embodiment, the first terpene-enriched cannabis composition is administered for day time and the second terpene-enriched cannabis composition is administered for night time.

According to another embodiment, the method for treating a patient comprises administering to the patient (i) a first terpene-enriched cannabis composition comprising a first cannabinoid at a first cannabinoid amount and a first primary terpene at a first primary terpene amount and (ii) a second terpene-enriched cannabis composition comprising the first cannabinoid at the first cannabinoid amount and a second primary terpenes at a second primary terpene amount.

According to an embodiment, the method for treating a patient comprises (i) administering to the patient multiple cannabis compositions to find the best working one; (ii) mimicking the best working composition by extracting cannabis plant material to form an extract and blending the extract with suitable terpenes, whereby a mimicking composition is formed and (iii) administering to the patient the mimicking composition. According to an embodiment, the method further comprises removing terpenes from the extract prior to the blending.

According to an embodiment, further provided is a method for treating conditions and/or symptoms selected from the group consisting of ulcer, viral infection, bacterial infection, fungal infection, infectious disease, and inflammation and combinations thereof, the method comprising administering to a subject in need thereof a therapeutically effective amount of a composition comprising (i) at least one primary terpene in a specific amount; (ii) at least 5% by weight of a non-cannabinoid, non-terpene carrier; (iii) optionally at least two secondary terpenes; and (iv) optionally at least one cannabinoid in a specific amount.

According to an embodiment, further provided is a composition comprising (i) at least one primary terpene in a specific amount; (ii) at least 5% by weight of a non-cannabinoid, non-terpene carrier; (iii) optionally at least two secondary terpenes; and (iv) optionally at least one cannabinoid in a specific amount, for use in treating conditions and/or symptoms selected from the group consisting of ulcer, viral infection, bacterial infection, fungal infection, infectious disease, and inflammation and combinations thereof.

According to an embodiment, further provided is the use of a composition comprising (i) at least one primary terpene in a specific amount; (ii) at least 5% by weight of a non-cannabinoid, non-terpene carrier; (iii) optionally at least two secondary terpenes; and (iv) optionally at least one cannabinoid in a specific amount, in the manufacture of a medicament for treating conditions and/or symptoms selected from the group consisting of ulcer, viral infection, bacterial infection, fungal infection, infectious disease, and inflammation and combinations thereof.

According to an embodiment, the composition comprises less than 20% by weight water.

According to an embodiment, further provided is a method for treating conditions and/or symptoms selected from the group consisting of ulcer, viral infection, bacterial infection, fungal infection and inflammation and combinations thereof, the method comprising administering to a subject in need thereof a therapeutically effective amount of a composition comprising (i) at least one primary terpene in a specific amount; (ii) at least 5% by weight of a non-cannabinoid, non-terpene carrier; (iii) optionally at least two secondary terpenes; and (iv) optionally at least one cannabinoid in a specific amount.

According to an embodiment, further provided is a composition comprising (i) at least one primary terpene in a specific amount; (ii) at least 5% by weight of a non-cannabinoid, non-terpene carrier; (iii) optionally at least two secondary terpenes; and (iv) optionally at least one cannabinoid in a specific amount for use in the treatment of conditions and/or symptoms selected from the group consisting of ulcer, viral infection, bacterial infection, fungal infection and inflammation and combinations thereof.

According to an embodiment, further provided is the use of a composition comprising (i) at least one primary terpene in a specific amount; (ii) at least 5% by weight of a non-cannabinoid, non-terpene carrier; (iii) optionally at least two secondary terpenes; and (iv) optionally at least one cannabinoid in a specific amount in the treatment of conditions and/or symptoms selected from the group consisting of ulcer, viral infection, bacterial infection, fungal infection and inflammation and combinations thereof.

According to an embodiment, the composition comprises less than 20% by weight water.

According to an embodiment, the conditions and/or symptoms are selected from the group consisting of skin ulcer, skin viral infection, skin bacterial infection, skin fungal infection, skin infectious disease and skin inflammation and combinations thereof.

According to an embodiment, the composition is for administration in a vaporizer. According to an embodiment, the composition is for sublingual administraion. According to an embodiment, the composition is for topical application. According to an embodiment, the composition is for administration as a suppository (vaginal or rectal). According to an embodiment, the composition is for administration as a mouthwash. According to an embodiment, the composition is for administration as a varnish.

According to an embodiment, the conditions and/or symptoms are selected from the group consisting of fungal infection, dermatophytes, candida infection, athlete's foot (Tinea pedis), yeast infection, jock itch (Tinea cruris), ringworm (Tinea Corporis), Tinea Capitis, Onychomycosis, Tinea Versicolor, Tinea Cruris, viral infection, Herpes, Herpes simplex, Herpes Zoster, Hand Foot and Mouth disease, Molluscum contagiosum, Viral Warts, Chickenpox, Measles, Rubella, bacterial infection, acne, Methycillin-Resistant Staphylococcus Aureus (MRSA), Necrotizing Fasciitis, Staphylococcal Scalded Skin Syndrome (SSSS), Leprosy, Cellulitis, Erysipelas, ulcer, pressure ulcer, diabetic ulcer, oral ulcer, mucositis, dermatitis, eczema, seborrhea, Lichen planus, periodontal disease, leishmaniasis, Scabies, an inflammatory injury, psoriasis, local pain, local itch and/or local burn, Temporomandibular-Joint (TMJ) and combination thereof.

According to an embodiment, the conditions and/or symptoms are selected from the group consisting of fungal infection, athlete's foot (Tinea pedis), yeast infection, jock itch (Tinea cruris), ringworm (Tinea Corporis), Tinea Capitis, Onychomycosis, Tinea Versicolor, Tinea Cruris, viral infection, Herpes, Herpes simplex, Herpes Zoster, Hand Foot and Mouth disease, Molluscum contagiosum, Viral Warts, Chickenpox, Measles, Rubella, bacterial infection, Necrotizing Fasciitis, Staphylococcal Scalded Skin Syndrome (SSSS), Leprosy, Cellulitis, Erysipelas, ulcer, pressure ulcer, diabetic ulcer, oral ulcer, mucositis, dermatitis, eczema, seborrhea, Lichen planus, periodontal disease, Scabies, an inflammatory injury, local pain, Temporomandibular-Joint (TMJ) and combinations thereof.

According to an embodiment, the conditions and/or symptoms are selected from the group consisting of fungal infection, athlete's foot (Tinea pedis), yeast infection, jock itch (Tinea cruris), ringworm (Tinea Corporis), Tinea Capitis, Onychomycosis, Tinea Versicolor, Tinea Cruris, viral infection, Herpes, Herpes simplex, Herpes Zoster, Hand Foot and Mouth disease, Molluscum contagiosum, Viral Warts, Chickenpox, Measles, Rubella, bacterial infection, Necrotizing Fasciitis, Staphylococcal Scalded Skin Syndrome (SSSS), Leprosy, Cellulitis, Erysipelas, ulcer, pressure ulcer, diabetic ulcer, oral ulcer, mucositis, dermatitis, eczema, seborrhea, Lichen planus, periodontal disease, Scabies, an Temporomandibular-Joint (TMJ) and combinations thereof.

According to an embodiment, the therapeutic effect comprises an increased immune response.

According to an embodiment, the at least one primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, geranyl acetate, camphor, menthol, iso-menthone, neral, gerial, viridiflorol, germacrene, thymol, Menth-2-en-1-ol, farensol, carotol, myrtenol and combinations thereof.

According to an embodiment, the composition comprises less than 5% by weight glycol.

According to an embodiment, the composition comprises less than 20% by weight water.

According to an embodiment, the composition has a therapeutic effect in treating conditions and/or symptoms associated with at least one selected from the group consisting of ulcer, viral infection, bacterial infection, fungal infection, infectious disease and inflammation and combinations thereof, wherein the therapeutic effect is an enhanced therapeutic effect compared with that of a composition comprising one half the amount of the at least one primary terpene.

According to an embodiment, the enhanced therapeutic effect is selected from the group consisting of a shortened onset time of said therapeutic effect, an increased magnitude of said therapeutic effect, an extended duration of said therapeutic effect, a reduced requirement for the composition, a reduction in at least one secondary adverse symptom, a reduced frequency of at least one of said conditions and/or symptoms, a reduced severity of at least one of said conditions and/or symptoms, a reduced consumption of other drugs and combinations thereof.

According to an embodiment, the shortened onset time of the therapeutic effect is a time at least 20% shorter than of the onset time obtained by administering a composition comprising one half the amount of the at least one primary terpene, in some embodiments at least 30%, at least 40%, at least 50% or at least 60% shorter.

According to an embodiment, the increased magnitude of the therapeutic effect is a magnitude at least 20% greater that the magnitude obtained by administering a composition comprising one half the amount of the at least one primary terpene, in some embodiments at least 30%, at least 40%, at least 50% at least 60%, at least 70%, at least 80%, at least 90% or at least 100% greater. According to an embodiment, the extended duration of the therapeutic effect is a duration of at least 20% longer than the duration obtained by administering a composition comprising one half the amount of the at least one primary terpene, in some embodiments at least 30%, at least 40%, at least 50% at least 60%, at least 70%, at least 80%, or at least 90% longer. According to an embodiment, the reduced requirement for the composition is a reduction of at least 20% in the dosage required to obtain the same therapeutic effect as that obtained by administering a dosage of a composition comprising one half the amount of the at least one primary terpene, in some embodiments a reduction of at least 30%, at least 40%, at least 50% at least 60%, at least 70%, at least 80%, or at least 90%. According to an embodiment, the reduction in at least one secondary adverse symptom is a decrease of at least 20% in a frequency, extent or severity of said adverse symptoms compared with a frequency, extent or severity of said adverse symptoms obtained by administering a dosage of a composition comprising one half the amount of the at least one primary terpene, in some embodiments a reduction of at least 30%, at least 40%, at least 50% at least 60%, at least 70%, at least 80%, or at least 90%. According to an embodiment, the reduced frequency of the conditions and/or symptoms is a reduction of at least 20% compared with the frequency of the conditions and/or symptoms obtained by administering a composition comprising one half the amount of the at least one primary terpene, in some embodiments a reduction of at least 30%, at least 40%, at least 50% at least 60%, at least 70%, at least 80%, or at least 900%. According to an embodiment, the reduced severity of the conditions and/or symptoms is a reduction at least 20% compared with a severity of the conditions and/or symptoms obtained by administering a composition comprising one half the amount of the at least one primary terpene, in some embodiments a reduction of at least 30%, at least 40%, at least 50% at least 60%, at least 70%, at least 80%, or at least 90%. According to an embodiment, the reduced consumption of other drugs is a reduction of at least 20% in the amount or frequency of other drugs consumed in order to obtain the same therapeutic effect compared with that obtained by administering a composition comprising one half the amount of the at least one primary terpene, in some embodiments a reduction of at least 30%, at least 40%, at least 50% at least 60%, at least 70%, at least 80%, or at least 900%.

According to an embodiment, the at least one primary terpene is selected from the group consisting of at least two, at least three, at least four or at least five of pinene, limonene, caryophyllene, caryophyllene oxide, nerolidol, eucalyptol, terpinene, terpineol, myrcene, cycloartenol, amyrin, camphene, cymene, ocimene, humulene, farnesene, guaiol, eudesmol, friedelin, linalool, camphor, menthol, thymol, carvacrol, eugenol, farnesol, citronellol, geraniol, citral and combinations thereof and the conditions and/or symptoms comprise a fungal infection. According to an embodiment, the conditions and/or symptoms comprise dermatophytes. According to an embodiment, the conditions and/or symptoms comprise athlete's foot (Tinea pedis). According to an embodiment, the conditions and/or symptoms comprise a yeast infection. According to an embodiment, the conditions and/or symptoms comprise jock itch (Tinea cruris). According to an embodiment, the conditions and/or symptoms comprise ringworm (Tinea Corporis). According to an embodiment, the conditions and/or symptoms comprise Tinea Capitis. According to an embodiment, the conditions and/or symptoms comprise Onychomycosis. According to an embodiment, the conditions and/or symptoms comprise Tinea Versicolor. According to an embodiment, conditions and/or symptoms comprise Tinea Cruris. According to an embodiment, the at least one primary terpene is selected from the group consisting of limonene, pinene, caryophyllene, caryophyllene oxide, nerolidol, eucalyptol and combinations thereof. According to an embodiment, the at least one primary terpene is selected from the group consisting of limonene, pinene, caryophyllene, caryophyllene oxide, eucalyptol and combinations thereof

According to an embodiment, the at least one primary terpene is selected from the group consisting of at least two, at least three, at least four or at least five of pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral and the conditions and/or symptoms comprise a Candida infection. According to an embodiment, the at least one primary terpene is selected from the group consisting of limonene, pinene caryophyllene, caryophyllene oxide, nerolidol, eucalyptol and combinations thereof. According to an embodiment, the at least one primary terpene is selected from the group consisting of limonene, pinene, nerolidol, caryophyllene, caryophyllene oxide, and combinations thereof.

According to an embodiment, the at least one primary terpene is selected from the group consisting of at least two, at least three, at least four or at least five of sabine, eucalyptol, pinene, borneol, caryophyllene, geraniol, limonene, myrcene, cycloartenol, citronellol, humulene, linalool, eudesmol, camphor, menthol and the conditions and/or symptoms comprise a Viral infection. According to an embodiment, the conditions and/or symptoms comprise Molluscum contagiosum. According to an embodiment, the conditions and/or symptoms comprise Viral Warts. According to an embodiment, the conditions and/or symptoms comprise Chickenpox. According to an embodiment, the conditions and/or symptoms comprise Measles. According to an embodiment, the conditions and/or symptoms comprise Rubella. According to an embodiment, the at least one primary terpene is selected from the group consisting of eucalyptol, caryophyllene, pinene, borneol, linalool and combination thereof.

According to an embodiment, the at least one primary terpene is selected from the group consisting of at least two, at least three, at least four or at least five of geraniol, eucalyptol, pinene, borneol, caryophyllene, terpineol, limonene, myrcene, cycloartenol, citronellol, humulene, linalool, eudesmol, camphor, menthol and the conditions and/or symptoms comprise Herpes, optionally Herpes simplex, and/or Herpes Zoster. According to an embodiment, the at least one primary terpene is selected from the group consisting of geraniol, eucalyptol, caryophyllene, pinene, terpineol, borneol, linalool, limonene and combination thereof. According to an embodiment, the at least one primary terpene is selected from the group consisting of geraniol, caryophyllene, pinene, terpineol, limonene and combination thereof.

According to an embodiment, the at least one primary terpene is selected from the group consisting of at least two, at least three, at least four or at least five of eucalyptol, citronellol, pinene, borneol, caryophyllene, geraniol, limonene, myrcene, cycloartenol, citronellol, humulene, linalool, eudesmol, camphor, menthol and the conditions and/or symptoms comprise Hand Foot and Mouth disease. According to an embodiment, the at least one primary terpene is selected from the group consisting of eucalyptol, caryophyllene, pinene, borneol, linalool and combination thereof.

According to an embodiment, the at least one primary terpene is selected from the group consisting of at least two, at least three, at least four or at least five of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, fenchol, geranyl acetate, camphor, menthol and the conditions and/or symptoms comprise Bacterial infection. According to an embodiment, the conditions and/or symptoms comprise Necrotizing Fasciitis. According to an embodiment, the conditions and/or symptoms comprise Staphylococcal Scalded Skin Syndrome (SSSS). According to an embodiment, the conditions and/or symptoms comprise Leprosy. According to an embodiment, the conditions and/or symptoms comprise Cellulitis. According to an embodiment, the conditions and/or symptoms comprise Erysipelas. According to an embodiment, the at least one primary terpene is selected from the group consisting of pinene, limonene, eucalyptol, caryophyllene, amyrin, linalool and combination thereof.

According to an embodiment, the at least one primary terpene is selected from the group consisting of at least two, at least three, at least four or at least five of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, camphor, menthol and the conditions and/or symptoms comprise acne. According to an embodiment, the at least one primary terpene is selected from the group consisting of limonene, linalool, pinene, terpineol, caryophyllene and combination thereof.

According to an embodiment, the at least one primary terpene is selected from the group consisting of at least two, at least three, at least four or at least five of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, camphor, menthol and the conditions and/or symptoms comprise Methycillin-Resistant Staphylococcus Aureus (MRSA). According to an embodiment, the at least one primary terpene is selected from the group consisting of pinene, limonene, amyrin, eucalyptol, linalool and combination thereof.

According to an embodiment, the at least one primary terpene is selected from the group consisting of at least two, at least three, at least four or at least five of pinene, limonene, linalool, germacrene, caryophyllene, nerolidol, terpineol, terpinene, eucalyptol, myrcene, cirtonellol, humulene, eudesmol, camphor, menthol and the conditions and/or symptoms comprise ulcer, optionally pressure ulcer and/or diabetic ulcer. According to an embodiment, the at least one primary terpene is selected from the group consisting of pinene, limonene, linalool, germacrene, caryophyllene, eucalyptol and combination thereof.

According to an embodiment, the at least one primary terpene is selected from the group consisting of at least two, at least three, at least four or at least five of pinene, limonene, linalool, germacrene, caryophyllene, nerolidol, terpineol, terpinene, eucalyptol, myrcene, cirtonellol, humulene, eudesmol, camphor, menthol and the conditions and/or symptoms comprise oral ulcer and/or mucositis. According to an embodiment, the at least one primary terpene is selected from the group consisting of pinene, limonene, linalool, caryophyllene, eucalyptol and combination thereof.

According to an embodiment, the at least one primary terpene is selected from the group consisting of at least two, at least three, at least four or at least five of camphene, sabinene, eugenol, cirtonellol, gerniol, terpineol, pinene, limonene, linalool, germacrene, caryophyllene, nerolidol, terpinene, eucalyptol, myrcene, humulene, eudesmol, camphor, menthol and the conditions and/or symptoms are selected from the group consisting of dermatitis, eczema, and/or seborrhea and combinations thereof. According to an embodiment, the conditions and/or symptoms comprise Lichen planus. According to an embodiment, the at least one primary terpene is selected from the group consisting of linalool, camphene, sabinene, limonene, myrcene.

According to an embodiment, the at least tone primary terpene is selected from the group consisting of at least two, at least three, at least four or at least five of pinene, limonene, nerolidol, Cycloartenol, caryophyllene, caryophyllene oxide, amyrin, eucalyptol, myrcene, cirtonellol, humulene, linalool, farnesene, phellandrene, Cymenen, eudesmol, carvacrol, eugenol, camphor, menthol and the conditions and/or symptoms comprise a periodontal disease. According to an embodiment, the at least one primary terpene is selected from the group consisting of carvacrol, eugenol, pinene, eucalyptol, amyrin and combination thereof.

According to an embodiment, the at least one primary terpene is selected from the group consisting of at least two, at least three, at least four or at least five of linalool, nerolidol, pinene, limonene, germacrene, caryophyllene, terpineol, terpinene, eucalyptol, myrcene, cirtonellol, humulene, eudesmol, camphor, menthol and the conditions and/or symptoms comprise Leishmaniasis. According to an embodiment, the at least one primary terpene is selected from the group consisting of linalool, nerolidol, pinene, caryophyllene, eucalyptol and combination thereof.

According to an embodiment, the at least one primary terpene is selected from the group consisting of at least two, at least three, at least four or at least five of caryophyllene, nerolidol, pinene, eucalyptol, limonene, myrcene, cirtonellol, humulene, terpineol, terpinene, guaiol, friedelin, eudesmol, linalool, camphor, menthol and the conditions and/or symptoms comprise Malaria. According to an embodiment, the at least one primary terpene is selected from the group consisting of caryophyllene, nerolidol, pinene, eucalyptol, limonene and combination thereof.

According to an embodiment, the at least one primary terpene is selected from the group consisting of at least two, at least three, at least four or at least five of caryophyllene, nerolidol, pinene, eucalyptol, limonene, myrcene, cirtonellol, humulene, terpineol, terpinene, guaiol, friedelin, eudesmol, linalool, camphor, menthol and the conditions and/or symptoms comprise Scabies. According to an embodiment, the at least one primary terpene is selected from the group consisting of camphene, linalool, sabinene, limonene and combination thereof.

According to an embodiment, the at least one primary terpene is selected from the group consisting of at least two, at least three, at least four or at least five of myrcene, linalool, caryophyllene, eucalyptol, borneol, pinene, sabinene, terpineol, camphor, menthol and the conditions and/or symptoms comprise local pain, local itch and/or local burn. According to an embodiment, the conditions and/or symptoms comprise Temporomandibular-Joint (TMJ). According to an embodiment, the at least one primary terpene is selected from the group consisting of linalool, myrcene, borneol, pinene, eucalyptol and combination thereof.

According to an embodiment, the at least one primary terpene is selected from the group consisting of at least two, at least three, at least four or at least five of pinene, limonene, eucalyptol, caryophyllene myrcene, citronellol, humulene, terpinene, terpineol, guaiol, friedelin, eudesmol linalool, borneol, amyrin, cycolartenol, camphor, menthol and the conditions and/or symptoms are selected from the group consisting of an inflammatory injury, wound, sore and ulcer or combinations thereof. According to an embodiment, the at least one primary terpene is selected from the group consisting of myrcene, humulene, linalool, caryophyllene, eucalyptol and combination thereof.

According to an embodiment, the at least one primary terpene is selected from the group consisting of at least two, at least three, at least four or at least five of linalool, caryophyllene, limonene, eucalyptol, myrcene, cirtonellol, pinene, sabinene, terpineol, terpinene, camphor, menthol and the conditions and/or symptoms comprise Psoriasis. According to an embodiment, the at least one primary terpene is selected from the group consisting of linalool, caryophyllene, limonene, sabinene, terpinene and combination thereof.

According to an embodiment, the at least one primary terpene is selected from the group consisting of at least two, at least three, at least four or at least five of terpinolene, linalool, amyrin, pinene, borneol, caryophyllene, geraniol and the therapeutic effect provides an increased immune response.

According to an embodiment, the at least one primary terpene is selected from the group consisting of at least two, at least three, or all four of terpinolene, linalool, amyrin, caryophyllene and combinations thereof and the therapeutic effect provides an increase immune response.

According to an embodiment, provided is a cannabinoid preparation comprising at least one cannabinoid at a concentration of about 10-50 mg/L, forming a preparation for treating conditions and/or symptoms associated with at least one of a fungal infection, bacterial infection, inflammation and/or ulceration. According to an embodiment, provided is a cannabinoid preparation comprising at least one cannabinoid at a concentration of at least 10 mg/L, at least 20 mg/L, at least 30 mg/L, at least 40 mg/L, or at least 50 mg/L. According to an embodiment, provided is a cannabinoid preparation comprising at least one cannabinoid at a concentration of less than 100 mg/L, less than 90 mg/L, less than 80 mg/L, less than 70 mg/L, less than 60 mg/L, less than 50 mg/L, less than 40, less than 30, less than 20, or less than 10 mg/L.

According to an embodiment, the cannabinoid preparation comprises an aqueous medium.

According to an embodiment, the cannabinoid preparation comprises at least one cannabinoid, wherein the at least one cannabinoid is selected from the group consisting of CBD, CBG, CBC, THC, CBDV, THCV, CBL, CBN and/or their acid form and combinations thereof. According to an embodiment, the cannabinoid preparation comprises CBD. According to an embodiment, the cannabinoid preparation comprises CBG. According to an embodiment, the cannabinoid preparation comprises CBC. According to an embodiment, the cannabinoid preparation comprises THC. According to an embodiment, the cannabinoid preparation comprises THCV. According to an embodiment, the cannabinoid preparation comprises CBDV. According to an embodiment, the cannabinoid preparation comprises CBL. According to an embodiment, the cannabinoid preparation comprises CBN.

According to an embodiment, the preparation further comprises at least one primary terpene, wherein said at least one terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, the preparation is for use in the treatment of a fungal infection, and said fungal infection comprises Candidiasis. According to an embodiment, the preparation is for use in the treatment of a fungal infection, and said fungal infection comprises Candidiasis albicans.

According to an embodiment, the preparation is for use in the treatment of inflammation and/or ulceration, and said inflammation and/or ulceration comprises Mucositis. According to an embodiment, the preparation is for use in the treatment of inflammation and/or ulceration, and said inflammation and/or ulceration comprises Esophagitis.

According to an embodiment, the preparation is for administration to patients selected from the group consisting of cancer patients, patients receiving chemotherapy, patients receiving hematopoietic stem cell transplantation patients receiving radiotherapy and patients with low immune response.

According to an embodiment, the preparation is for use in the treatment of conditions and/or symptoms selected from the group consisting of fungal infection, bacterial infection, inflammation and ulceration and combinations thereof, wherein said fungal infection, bacterial infection, inflammation or ulceration is an oral and/or vaginal infection.

According to an embodiment, provided is a method for treating conditions and/or symptoms selected from the group consisting of a fungal infection, bacterial infection, inflammation and/o ulceration and combinations thereof, the method comprising administrating a preparation comprising at least one cannabinoid at a concentration of about 10-50 mg/L.

According to an embodiment, provided a preparation comprising at least one cannabinoid at a concentration of about 10-50 mg/L for use in the treatment of conditions and/or symptoms selected from the group consisting of a fungal infection, bacterial infection, inflammation and/o ulceration and combinations thereof.

According to an embodiment, provided the use of a preparation comprising at least one cannabinoid at a concentration of about 10-50 mg/L in the manufacture of a medicament for the treatment of conditions and/or symptoms selected from the group consisting of a fungal infection, bacterial infection, inflammation and/o ulceration and combinations thereof.

According to an embodiment, the preparation for administration comprises at least one cannabinoid at a concentration of at least 10 mg/L, at least 20 mg/L, at least 30 mg/L, at least 40 mg/L, or at least 50 mg/L. According to an embodiment, provided is a cannabinoid preparation comprising at least one cannabinoid at a concentration of less than 100 mg/L, less than 90 mg/L, less than 80 mg/L, less than 70 mg/L, less than 60 mg/L, less than 50 mg/L, less than 40, less than 30, less than 20, or less than 10 mg/L.

According to an embodiment, the preparation comprises an aqueous medium.

According to an embodiment, the preparation comprises at least one cannabinoid, wherein said at least one cannabinoid comprises CBD, CBG, CBC, THC, CBDV, THCV, CBL, CBN and/or their acid form. According to an embodiment, the preparation comprises CBD. According to an embodiment, the preparation comprises CBG. According to an embodiment, the preparation comprises CBC. According to an embodiment, the preparation comprises THC. According to an embodiment, the preparation comprises THCV. According to an embodiment, the preparation comprises CBDV. According to an embodiment, the preparation comprises CBL. According to an embodiment, the preparation comprises CBN.

According to an embodiment, the preparation further comprises at least one terpene, wherein said at least one terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, the preparation is for use in the treatment of a fungal infection, and said fungal infection comprises Candidiasis. According to an embodiment, the preparation is for use in the treatment of a fungal infection, and said fungal infection comprises Candidiasis albicans.

According to an embodiment, the preparation is for use in the treatment of a inflammation and/or ulceration, and said inflammation and/or ulceration comprises Mucositis. According to an embodiment, the preparation is for use in the treatment of a inflammation and/or ulceration, and said inflammation and/or ulceration comprises Esophagitis.

According to an embodiment, the preparation is for administration to patients selected from the group consisting of cancer patients, patients receiving chemotherapy, patients receiving hematopoietic stem cell transplantation patients receiving radiotherapy and patients with low immune response and combinations thereof.

According to an embodiment, the preparation is for use in the treatment of a fungal infection, bacterial infection, inflammation and ulceration and combinations thereof, wherein said fungal infection, bacterial infection, inflammation or ulceration is an oral and/or vaginal infection.

### Examples 1-30

The Table presents examples of compositions as described herein.

| | | | | Content | | |
|---|---|---|---|---|---|---|
| | | | | (% by weight) | | |
| | | | | | Primary | Therapeutic ef [symbol] |
| | Form | Cannabinoid | Primary terpene | Cannabinoid | terpene | For: |
| 1 | Topical | CBD | Eucalyptol | 3-25 | 1-10 | Tinea Capitis |
| | | | | | | infection |
| 2 | Topical | CBG | Pinene | 3-25 | 1-10 | Candida infect [symbol] |
| 3 | Topical | CBC | Terpinene | 3-25 | 1-10 | yeast infection |
| 4 | Topical | THC | Linalool | 3-25 | 1-10 | Local pain |
| 5 | Topical | THC | Pinene | 3-25 | 1-10 | Measles |
| 6 | Topical | CBD | borneol | 3-25 | 1-10 | Herpes simple [symbol] |
| 7 | Topical | CBD | amyrin | 3-25 | 1-10 | Cellulitis |
| 8 | Topical | CBG | linalool | 3-25 | 1-10 | Scabies |
| 9 | Topical | CBN | Limonene | 3-25 | 1-10 | Pressure ulcer |
| 10 | Topical | CBD | Caryophyllene | 3-25 | 1-10 | Hand Foot and |
| | | | | | | Mouth disease |
| 11 | Topical | CBG | Eucalyptol | 3-25 | 1-10 | Herpes Zoster |
| 12 | Topical | CBD | Limonene | 3-25 | 1-10 | Acne |
| 13 | Topical | CBN | Terpinene | 3-25 | 1-10 | Methycillin- |
| | | | | | | Resistant |
| | | | | | | Staphylococc |
| | | | | | | us Aureus |
| | | | | | | (MRSA). |
| 14 | Mouthwash | CBC | Eucalyptol | 3-25 | 1-10 | Oral mucositis |
| 15 | Topical | CBG | Myrcene | 3-25 | 1-10 | Local pain |
| 16 | Topical | CBD | Caryophyellen | 3-25 | 1-10 | Diabetic ulcer |
| 17 | Topical | CBD | Sabinene | 3-40 | 1-20 | Eczema |
| 18 | Topical | THC | Sabinene | 3-40 | 1-20 | Psoriasis |
| 19 | Topical | CBD | Humulene | 3-40 | 1-20 | Dermatitis |
| 20 | Topical | CBD | Pinene | 3-40 | 1-20 | Periodontal dis [symbol] |
| 21 | Oral tablet | CBG | Limonene | 3-30 | 1-10 | Onychomycosi [symbol] |
| 22 | Suppository | CBL | Pinene and limonene | 3-25 | 1-10 | Systemic candi [symbol] |
| | | | | | | infection |
| 23 | Oral tablet | CBL | Eucalyptol, geraniol | 3-30 | 1-10 | Chickenpox |
| | | | and borneol | | | |
| 24 | Suppository | THCV and | Eusesmoland sabine. | 3-30 | 1-10 | Rubella |
| | | CBC | | | | |
| 25 | Suppository | CBD | Geraniol and pinene | 3-30 | 1-10 | Herpes |
| 26 | Mouthwash | THC | linalool | 3-30 | 1-10 | Cellulitis |
| 27 | Varnish | CBD and | Pinene and myrcene | 3-40 | 1-20 | Fungal |
| | | CBG | | | | infection of |
| | | | | | | nail |
| 28 | Suppository | CBDC | Amyrin | 3-40 | 1-20 | MRSA |
| 29 | Topical | CBD | Limonene | 3-30 | 1-10 | Diabetic ulcer |
| 30 | Mouthwash | CBN and | Germacrene and | 3-30 | 1-10 | Mucositis |
| | | CBLA | Caryophyllene | | | |

[symbol]

indicates data missing or illegible when filed

Thus, the scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for treating conditions and/or symptoms selected from the group consisting of ulcer, viral infection, bacterial infection, fungal infection, infectious disease, and inflammation comprising administering to a subject in need thereof a therapeutically effective amount of a composition comprising
(i) at least one cannabinoid in a specific amount,
(ii) at least one terpene in a specific amount, and
(iii) at least 5% by weight of a non-cannabinoid, non-terpene carrier
(a) wherein said non-cannabinoid, non-terpene carrier comprises cellulose and a weight/weight ratio of a total amount of terpenes to a total amount of cannabinoids in said composition is from about 0.1:1 to about 1:1, or;
(b) wherein said non-cannabinoid, non-terpene carrier comprises less than 5% by weight cellulose and a weight/weight ratio of a total amount of terpenes to a total amount cannabinoids in said composition is about 0.05:1 to about 1:1.

2. The method of claim 1, wherein said conditions and/or symptoms are selected from the group consisting of fungal infection, dermatophytes, candida infection, athlete's foot (Tinea pedis), yeast infection, jock itch (Tinea cruris), ringworm (Tinea Corporis), Tinea Capitis, Onychomycosis, Tinea Versicolor, Tinea Cruris, viral infection, Herpes, Herpes simplex, Herpes Zoster, Hand Foot and Mouth disease, Molluscum contagiosum, Viral Warts, Chickenpox, Measles, Rubella, bacterial infection, acne, Methycillin-Resistant Staphylococcus Aureus (MRSA), Necrotizing Fasciitis, Staphylococcal Scalded Skin Syndrome (SSSS), Leprosy, Cellulitis, Erysipelas, ulcer, pressure ulcer, diabetic ulcer, oral ulcer, mucositis, dermatitis, eczema, seborrhea, Lichen planus, periodontal disease, leishmaniasis, scabies, malaria, an inflammatory injury, psoriasis, local pain, local itch and/or local burn, Temporomandibular-Joint (TMJ) and combination thereof.

3. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of pinene, limonene, caryophyllene, caryophyllene oxide, nerolidol, eucalyptol, terpinene, terpineol, myrcene, cycloartenol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral and combinations thereof and wherein said conditions and/or symptoms comprise a fungal infection.

4. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral and combinations thereof and wherein said conditions and/or symptoms comprise Candida infection.

5. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of sabine, eucalyptol, pinene, borneol, caryophyllene, geraniol, limonene, myrcene, cycloartenol, citronellol, humulene, linalool, eudesmol, camphor, menthol and combinations thereof and wherein said conditions and/or symptoms comprise a viral infection.

6. The method of claim 1, wherein said at least one cannabinoid is selected from the group consisting of CBD, THC, CBC, CBG, CBN, CBDa and THCa, CBLA, THCV and combinations thereof, wherein said at least one terpene is selected from the group consisting of geraniol, eucalyptol, pinene, borneol, caryophyllene, terpineol, limonene, myrcene, cycloartenol, citronellol, humulene, linalool, eudesmol, camphor, menthol and combinations thereof and wherein said conditions and/or symptoms comprise herpes, optionally herpes simplex, and/or herpes zoster.

7. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of eucalyptol, citronellol, pinene, borneol, caryophyllene, geraniol, limonene, myrcene, cycloartenol, citronellol, humulene, linalool, eudesmol, camphor, menthol and combinations thereof and wherein said conditions and/or symptoms comprise Hand Foot and Mouth Disease.

8. The method of claim 1, wherein said at least cannabinoid is selected from the group consisting of CBD, THC, CBC, CBG, CBN, CBL, CBDV, THCV and combinations thereof, wherein said at least one terpene is selected from the group consisting of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, fenchol, geranyl acetate, camphor, menthol and combinations thereof and wherein said conditions and/or symptoms comprise a bacterial infection.

9. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, camphor, menthol and combinations thereof and wherein said conditions and/or symptoms comprise acne.

10. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of pinene, limonene, eucalyptol, farnesene, cymene, phellandrene, borneol, caryophyllene, geraniol, terpinene, terpineol, amyrin, camphor, sabine, myrcene, citronellol, humulene, guaiol, friedelin, eudesmol linalool, camphor, menthol and combinations thereof and wherein said conditions and/or symptoms comprise Methycillin-Resistant Staphylococcus Aureus (MRSA).

11. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of pinene, limonene, linalool, germacrene, caryophyllene, nerolidol, terpineol, terpinene, eucalyptol, myrcene, cirtonellol, humulene, eudesmol, camphor, menthol and combinations thereof and wherein said conditions and/or symptoms comprise an ulcer, optionally a pressure ulcer and/or diabetic ulcer.

12. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of pinene, limonene, linalool, germacrene, caryophyllene, nerolidol, terpineol, terpinene, eucalyptol, myrcene, cirtonellol, humulene, eudesmol, camphor, menthol and combinations thereof and wherein said conditions and/or symptoms comprise oral ulcer and/or mucositis.

13. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of camphene, sabinene, eugenol, cirtonellol, gerniol, terpineol, pinene, limonene, linalool, germacrene, caryophyllene, nerolidol, terpinene, eucalyptol, myrcene, humulene, eudesmol, camphor, menthol and combinations thereof and wherein said conditions and/or symptoms are selected from the group consisting of dermatitis, eczema, and seborrhea and combinations thereof.

14. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of pinene, limonene, nerolidol, Cycloartenol, caryophyllene, caryophyllene oxide, amyrin, eucalyptol, myrcene, cirtonellol, humulene, linalool, farnesene, phellandrene, Cymenen, eudesmol, carvacrol, eugenol, camphor, menthol and combinations thereof and wherein said conditions and/or symptoms comprise a periodontal disease.

15. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of linalool, nerolidol, pinene, limonene, germacrene, caryophyllene, terpineol, terpinene, eucalyptol, myrcene, cirtonellol, humulene, eudesmol, camphor, menthol and combinations thereof and wherein said conditions and/or symptoms comprise leishmaniasis.

16. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of caryophyllene, nerolidol, pinene, eucalyptol, limonene, myrcene, cirtonellol, humulene, terpineol, terpinene, guaiol, friedelin, eudesmol, linalool, camphor, menthol and combinations thereof and wherein said conditions and/or symptoms comprise malaria.

17. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of caryophyllene, nerolidol, pinene, eucalyptol, limonene, myrcene, cirtonellol, humulene, terpineol, terpinene, guaiol, friedelin, eudesmol, linalool, camphor, menthol and combinations thereof and wherein said conditions and/or symptoms comprise scabies.

18. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of pinene, limonene, eucalyptol, caryophyllene myrcene, citronellol, humulene, terpinene, terpineol, guaiol, friedelin, eudesmol linalool, borneol, amyrin, cycolartenol, camphor, menthol and combinations thereof and wherein said conditions and/or symptoms are selected from the group consisting of an inflammatory injury, wound, sore and ulcer.

19. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of linalool, caryophyllene, limonene, eucalyptol, myrcene, cirtonellol, pinene, sabinene, terpineol, terpinene, camphor, menthol and combinations thereof and wherein said conditions and/or symptoms comprise psoriasis.

20. The method of claim 1, wherein said at least one cannabinoid is selected from the group consisting of CBD, THC, CBC, CBG, CBN, CBL, CBDV, THCV and combinations thereof, wherein said at least one terpene is selected from the group consisting of myrcene, linalool, caryophyllene, eucalyptol, borneol, pinene, sabinene, terpineol, camphor, menthol and combinations thereof and wherein said conditions and/or symptoms are selected from the group consisting of local pain, local itch and local burn and combinations thereof.

21. The method of claim 1, comprising
(i) administering to said subject for a first period of time a first said composition comprising a first cannabinoid at a first cannabinoid amount and a first primary terpene at a first primary terpene amount,
followed by
(ii) administering to said subject for a second period of time a second said composition comprising said first cannabinoid at said first cannabinoid amount and a second primary terpene at a second primary terpene amount.

22. The method of claim 1, wherein said condition and/or symptoms are selected from the group consisting of a fungal infection, bacterial infection, inflammation and ulceration, said composition comprising at least one cannabinoid at a concentration of about 10-50 mg/L, wherein said preparation comprises an aqueous medium.
